(19) United States
(12) Patent Application Publication
(10) Pub. No.: US 2009/0074740 A1
(43) Pub. Date: Mar. 19, 2009

(54) Use of haptoglobin genotyping in diagnosis and treatment of defective reverse cholesterol transport (RCT)

(76) Inventors: Noah Berkowitz, New Rochelle, NY (US); Andrew Levy, Haifa (IL)

Correspondence Address: Pearl Cohen Zedek Latzer, LLP, 1500 Broadway, 12th Floor, New York, NY, 10036, US

(21) Appl. No.: 12/153,186

(22) Filed: May 14, 2008

Related U.S. Application Data
(60) Provisional application No. 60/924,412, filed on May 14, 2007.
(60) Provisional application No. 60/924,723, filed on May 29, 2007.
(60) Provisional application No. 60/996,552, filed on Nov. 23, 2007.

(51) Int. Cl. A61K 38/44; C12Q 1/68; A61K 31/355; A61K 31/35

(52) U.S. Cl. 424/94.4; 435/6; 514/458; 514/460

(57) ABSTRACT

A method of determining a potential of a nondiabetic or diabetic patient to benefit from reverse cholesterol transport therapy for treatment of a vascular complication, followed by methods and compositions of treating the diagnosed vascular complications comprising determining a haptoglobin phenotype of the patient. Reverse cholesterol transport therapy includes inhibition of cholesteryl ester transport protein, such as by using the compound torcetrapib.

### CROSS-REFERENCE TO RELATED APPLICATIONS

This application claims priority to U.S. provisional application Ser. No. 60/924,412, filed May 14, 2007, Ser. No. 60/924,723, filed May 29, 2007, and Ser. No. 60/996,552, filed Nov. 23, 2007, all three of which are incorporated herein by reference in their entireties.

### FIELD OF THE INVENTION

The present invention relates to methods of determining the prospective benefits of reverse cholesterol transport therapy, and in particular utility of cholesteryl ester transfer protein inhibitors, for prevention of cardiovascular disease in individuals, based on polymorphism at the haptoglobin 2 allele and subsequent therapies.

### BACKGROUND OF THE INVENTION

Atherosclerosis, the accumulation of cholesterol in the arteries that clogs the circulation and results in heart attacks and strokes, is a leading cause of death. One strategy for preventing heart disease and stroke is to clear out clogged arteries, restoring circulation. This process, known as reverse cholesterol transport is accomplished by the high-density lipoproteins (HDLs) in the blood. HDL transports excess cholesterol from the artery wall and macrophages and delivers it to the liver, where it is excreted as bile salts and cholesterol. One method for boosting reverse cholesterol transport has been to elevate plasma HDL levels by inhibiting a protein called CETP that transfers cholesterol esters from HDL to lower-density lipoproteins.

Haptoglobin (Hp) is a hemoglobin-binding serum protein which plays a major role in the protection against heme-driven oxidative stress. Mice lacking the Hp gene demonstrate a dramatic increase in oxidative stress and oxidative tissue damage particularly in the kidney. In man, there are two common alleles for Hp (1 and 2) manifesting as three major phenotypes 1-1, 2-1 and 2-2. Functional differences in the hemoglobin-binding capacity of the three phenotypes have been demonstrated. Hp in patients with the Hp 1-1 phenotype is able to bind more hemoglobin on a per gram basis than Hps containing products of the haptoglobin 2 allele. Haptoglobin molecules in patients with the haptoglobin 1-1 phenotype are also more efficient antioxidants, since the smaller size of haptoglobin 1-1 facilitates its entry to extravascular sites of oxidative tissue injury compared to products of the haptoglobin 2 allele. This also includes a significantly greater glomerular sieving of haptoglobin in patients with haptoglobin 1-1.

The haptoglobin 2 allele appears to have arisen from the 1 allele via a partial gene duplication event approximately 20 million years ago and to have spread in the world population as a result of selective pressures related to resistance to infectious agents. Presently the haptoglobin alleles differ dramatically in their relative frequency among different ethnic groups. The gene duplication event has resulted in a dramatic change in the biophysical and biochemical properties of the haptoglobin protein encoded by each of the 2 alleles. For example, the protein product of the 1 allele appears to be a superior antioxidant compared to that produced by the 2 allele. The haptoglobin phenotype of any individual, 1-1, 2-1 or 2-2, is readily determined from 10 microliters of plasma by gel electrophoresis.

It was demonstrated that the haptoglobin phenotype is predictive of the development of a number of microvascular complications in the diabetic patient. Specifically, it was shown that patients who are homozygous for the haptoglobin 1 allele are at decreased risk for developing retinopathy and nephropathy. This effect, at least for nephropathy, has been observed in both type 1 and type 2 diabetic patients and the relevance strengthened by the finding of a gradient effect with respect to the number of haptoglobin 2 alleles and the development of nephropathy. Furthermore, it was shown that the haptoglobin phenotype may be predictive of the development of macrovascular complications in the diabetic patient. The development of restenosis after percutaneous coronary angioplasty is significantly decreased in diabetic patients with the 1-1 haptoglobin phenotype. Previous retrospective and cross-sectional studies examining haptoglobin phenotype and coronary artery disease in the general population have yielded conflicting results.

Some prior art publications teach methods of correlating haptoglobin phenotype and disease. WO98/37419 teaches a method and kit for determining a haptoglobin phenotype and specifically relates to applications involving human haptoglobin. Teachings of this application focus on use of the haptoglobin 2-2 phenotype as an independent risk factor, specifically in relation to target organ damage in refractory essential hypertension, in relation to atherosclerosis (in the general population) and acute myocardial infarction and in relation to mortality from HIV infection.

CETP is a glycoprotein physically associated with HDL particles. It helps transport cholesterol ester from HDL to lipoproteins that contain apolipoprotein B. This process is followed by the transference of triglycerides in the opposite direction. Torcetrapib (CP-529414, Pfizer) is a drug being developed to treat hypercholesterolemia (elevated cholesterol levels) and prevent cardiovascular disease. It acts by inhibiting cholesteryl ester transfer protein (CETP), resulting in higher HDL cholesterol levels (the “good” cholesterol-containing particle) and reducing LDL cholesterol levels (the “bad” cholesterol).

There is a widely recognized need for, and it would be highly advantageous to have a method to predict which patients with cardiovascular disease would benefit from reverse cholesterol transport therapies, such as CETP inhibitors including torcetrapib. Such a method would allow medical practitioners to maximize therapeutic benefit while minimizing risk to each patient to the greatest possible extent.

Likewise, it would be highly advantageous to augment the therapy of those patients diagnosed to benefit from reverse cholesterol transport therapies, with compounds known to aid in the reduction of oxidative stress.

### SUMMARY OF THE INVENTION

In one embodiment, provided herein is a method of determining prognosis for a subject having a vascular complication to benefit from treatment with reverse cholesterol transport therapy comprising the step of obtaining a biological sample from the subject; and determining the subject's haptoglobin allelic genotype, whereby a subject expressing the Hp-2-2 genotype will benefit from treatment with reverse cholesterol transport therapy, such as but not limited to CETP inhibitor therapy, elevating plasma HDL levels more than Hp-2-1 and Hp-1-1 genotypes.

In another embodiment, the invention provides a method of treating a subject having a vascular complication, comprising the step of contacting the subject with an effective amount of a composition comprising glutathione peroxidase or its mimetic, isomer, metabolite, and/or salt therefore, and cholesteryl ester transfer protein inhibitor thereby treating vascular complication.

In one embodiment, provided herein is a method of inhibiting or suppressing a vascular complication in a subject comprising the step of contacting the subject with an effective amount of a composition comprising glutathione peroxidase or its mimetic, isomer, metabolite, and/or salt therefore, and cholesteryl ester transfer protein inhibitor thereby inhibiting or suppressing vascular complication.

In another embodiment, provided herein is a method of reducing symptoms associated with a vascular complication in a subject comprising the step of contacting the subject with an effective amount of a composition comprising glutathione peroxidase or its mimetic, isomer, metabolite, and/or salt therefore, and cholesteryl ester transfer protein inhibitor thereby reducing symptoms associated with vascular complication.

In one embodiment, provided herein is a composition for treating a vascular complication in a subject comprising: a therapeutically effective amount of a composition comprising glutathione peroxidase or its mimetic, isomer, metabolite, and/or salt therefore and cholesteryl ester transfer protein inhibitor.

In another embodiment, there is provided a method of determining the importance of treating a diabetic patient with abnormal or impaired cholesterol efflux with an antioxidant, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method of determining the importance of treating a diabetic patient with abnormal or impaired cholesterol efflux with an antioxidant so as to prevent a diabetes-associated vascular complication, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method of determining the importance of treating a diabetic patient with abnormal or impaired macrophage cholesterol efflux with an antioxidant, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method of determining the importance of treating a diabetic patient with abnormal or impaired macrophage cholesterol efflux with an antioxidant so as to prevent a diabetes-associated vascular complication, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method of determining the importance of treating a diabetic patient with an abnormal, defective or impaired reverse cholesterol transport with an antioxidant, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method of determining the importance of treating a diabetic patient with an abnormal or impaired reverse cholesterol transport with an antioxidant so as to prevent a diabetes-associated vascular complication, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-phenotypes.

In another embodiment, there is provided a method of determining the importance of treating a diabetic patient with an abnormal or impaired hypercholesterolemia with an antioxidant, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method of determining the importance of treating a diabetic patient with an abnormal or impaired hypercholesterolemia with an antioxidant so as to prevent a diabetes-associated vascular complication, the method comprising the step of determining a haptoglobin phenotype of the diabetic patient, thereby determining the importance of reducing the oxidative stress in the specific diabetic patient, wherein the importance is greater in a patient having a haptoglobin 2-2 phenotype compared to patients having haptoglobin 1-2 phenotype or haptoglobin 1-1 phenotypes.

In another embodiment, there is provided a method for correcting abnormal or impaired cholesterol efflux in a diabetic patient, the method comprising the step of determining a haptoglobin phenotype of the diabetic patient, wherein ability to provide the correcting is greater in a patient having a haptoglobin 2-2 phenotype compared to patients having haptoglobin 1-2 phenotype or haptoglobin 1-1 phenotypes, and correcting the abnormal or impaired cholesterol efflux by administering an antioxidant.

In another embodiment, there is provided a method for correcting abnormal or impaired macrophage cholesterol efflux in a diabetic patient, the method comprising the step of determining a haptoglobin phenotype of the diabetic patient, wherein ability to provide the correcting is greater in a patient having a haptoglobin 2-2 phenotype compared to patients having haptoglobin 1-2 phenotype or haptoglobin 1-1 phenotypes, and correcting the abnormal or impaired macrophage cholesterol efflux by administering an antioxidant.

In another embodiment, there is provided a method for correcting an abnormal or impaired reverse cholesterol transport in a diabetic patient, the method comprising the step of determining a haptoglobin phenotype of the diabetic patient, wherein ability to provide the correcting is greater in a patient having a haptoglobin 2-2 phenotype compared to patients having haptoglobin 1-2 phenotype or haptoglobin 1-1 phenotypes, and correcting the abnormal or impaired reverse cholesterol transport is achieved by administering an antioxidant.

In another embodiment, there is provided a method for correcting hypercholesterolemia in a diabetic patient, the method comprising the step of determining a haptoglobin phenotype of the diabetic patient, wherein ability to provide the correcting is greater in a patient having a haptoglobin 2-2 phenotype compared to patients having haptoglobin 1-2 phenotype or haptoglobin 1-1 phenotypes, and correcting the hypercholesterolemia is achieved by administering an antioxidant.

According to further features in embodiments described below, the vascular complication is selected from the group consisting of a microvascular complication and a macrovascular complication.

According to yet further features in embodiments described below, the vascular complication is a macrovascular complication selected from the group consisting of atherosclerosis, coronary artery disease, chronic heart failure, cardiovascular death, stroke, myocardial infarction and coronary angioplasty associated restenosis.

According to still further features in embodiments described below, the microvascular complication is selected from the group consisting of diabetic retinopathy, diabetic nephropathy and diabetic neuropathy.

According to further features in embodiments described below, the macrovascular complication is selected from the group consisting of fewer coronary artery collateral blood vessels and myocardial ischemia.

According to further features in embodiments described below, antioxidants can include antioxidant vitamins such as but not limited to vitamin E and vitamin C, glutathione peroxidase mimetics, and other antioxidant compounds such as ramipril and probucol.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description

### BRIEF DESCRIPTIONS OF THE FIGURES

The invention will be better understood from a reading of the following detailed description taken in conjunction with the drawings in which like reference designators are used to designate like elements, and in which:

FIG. 1 depicts cholesterol efflux from J774 macrophages incubated with serum from DM and non-DM individuals. Serum HDL-mediated cholesterol efflux normalized for HDL was determined as described in Materials and Methods. Results are presented as the mean±SEM. The rate of cholesterol efflux was both Hp genotype and DM dependent. There was a significantly increased cholesterol efflux from macrophages incubated with serum from non-DM individuals as compared with serum from DM individuals (P<0.001). In the DM group, there was significantly increased cholesterol efflux from macrophages incubated with serum from DM Hp1-1 patients as compared with DM Hp2-1 or Hp2-2 patients (10.2±1.1%, 7.5±1.2%, 6.6±0.8% efflux rate for Hp1-1, Hp2-1, and Hp2-2, respectively; P<0.001; n=30 for each group);

FIG. 2 depicts the measurement of LCAT cholesterol esterification rate in the serum of DM and non-DM individuals segregated by the Hp type. LCAT cholesterol esterification rate (FER per hour±SEM) normalized for HDL was assessed in the serum of 84 diabetic and 62 nondiabetic individuals with Hp1-1, Hp2-1, and Hp2-2. There was a significant reduction in LCAT cholesterol esterification rate in the DM group exclusively in Hp2-1 and Hp2-2 sera (FER per hour: 2.10±0.46, 1.48±0.39, 1.13±0.18 for Hp1-1 [n=23], Hp2-1 [n=28], and Hp2-2 [n=33], respectively; P<0.01);

FIG. 3 depicts HDL-mediated cholesterol efflux from J774 cells incubated with normal or glycated Hb with and without Hp. Cholesterol efflux obtained using purified HDL incubated with J774 macrophage cells was taken as 100%, and the effect of Hb (native and glycated) and Hp on this efflux was studied. Data shown represent the mean±SEM of 4 independent experiments. Hp and native Hb had no effect on efflux. However, glycated Hb significantly reduced this efflux (34±3%, P<0.001). The reduction in cholesterol efflux by glycated Hb was blocked to a significantly greater degree with Hp1-1 as compared with Hp2-2 (80±6% vs 30±4%, P<0.001);

FIG. 4 depicts Hp- and DM-dependent differences in RCT in vivo. C57BL/6 mice were injected with ³H-cholesterol-labeled and cholesterol-loaded J774 foam cells and monitored for the presence of ³H-tracer in plasma, liver, and feces for 48 hours, as described in Materials and Methods. A, ³H-Cholesterol in plasma at 24 and 48 hours. Data shown represent the mean±SEM of 4 mice for each group. Data are expressed as counts per minute in 1 mL of plasma. There was a 54±9% reduction in plasma ³H-cholesterol in DM Hp2 mice and a 25±13% reduction in DM Hp1 mice (P<0.03) as compared with non-DM mice. B, ³H-Cholesterol in liver at 48 hours. Data shown represent the mean±SEM of 4 mice for each group. Data are expressed as counts per minute in 100 mg of liver tissue. There was a 52±10% reduction in liver ³H-cholesterol in DM Hp2 mice and a 27±14% reduction in DM Hp1 mice (P<0.03) as compared with non-DM mice. C, ³H-Cholesterol and ³H-bile acids at 48 hours. Data shown represent the mean±SEM of 4 mice for each group. Data are expressed as counts per minute in total feces collected continuously from 0 to 48 hours. There were no significant differences in the amount of ³H-total sterols, ³H-cholesterol, or ³H-bile acids excreted by non-DM Hpl mice compared with non-DM Hp2 mice (P<0.3), whereas in the DM Hpl mice, there was 1.7-fold greater ³H-total sterols, 1.8-fold greater ³H-cholesterol, and 1.6-fold greater ³H-bile acids compared with DM Hp2 mice. All of these differences, with the exception of ³H-bile acids, were statistically significant (P<0.03). The reduction in ³H-tracer in feces associated with DM in these mice was only significant in the Hp2 group (53±11% less ³H-total sterol and 57±10% less ³H-cholesterol compared with non-DM Hp2 mice; P<0.03);

FIG. 5 shows the half life of the Hp 2-Hb complex is increased compared to the Hp 1-Hb complex in non-diabetic mice and rats. The half-life of ¹²⁵I-labeled Hp 1-Hb and Hp 2-Hb complex was measured in (A) Hp 1 mice, (B) Hp 2 mice and (C) rats (Hp 1) as described in methods. The mean half-lives and number of animals used in each group is supplied in Table 2. The half-life of the Hp 2-Hb complex was significantly increased as compared to the Hp 1-Hb complex in all animals and strains studied (p<0.0001);

FIG. 6 shows the half-life of the Hp 2-Hb complex but not the Hp 1-Hb complex is increased in DM. The half-life of ¹²⁵I-labeled Hp 1-Hb and Hp 2-Hb complex was measured in Diabetic Hp 1 mice or Diabetic Hp 2 mice (not shown) as described in methods. The mean half-lives and number of mice used in each group is supplied in Table 3. The half-life of the Hp 2-Hb complex was significantly increased in Hp 2 DM mice compared to that observed in Hp 2 mice without DM (103±9.5 min vs. 18.6±4.5 min, n=6, p<0.0001). DM had no effect on the half-life of the Hp 1-Hb complex;

FIG. 7 shows increased association of the Hp 2-Hb complex with HDL in DM. Immunoprecipitation of HDL was performed as described in methods 75 minutes after mice were injected with ¹²⁵I-labeled Hp 1-Hb or Hp 2-Hb complex. Shown in the figure is the percentage of cpm in the serum coimmunoprecipitated with HDL in Hp 2 mice at this 75 minute time point. The type of Hp-Hb complex and DM were both independently associated with the fraction of complex in the serum associated with HDL. In the sera of mice injected with Hp 2-Hb complex a significantly higher percentage of the complex was associated with HDL. Furthermore, in the sera of DM mice injected with Hp 1-Hb or Hp 2-Hb complex a significantly higher percentage of the complex was associated with HDL; and

FIG. 8 shows impaired cholesterol efflux from J774 macrophages incubated with serum from Hp 2 DM mice and prevention of this impairment with vitamin E. Efflux (%/hour) normalized for HDL concentration was determined as described in methods. The rate of cholesterol efflux was both Hp genotype and DM dependent. In mice without DM there was no difference in efflux elicited by serum from Hp 1 or Hp 2 mice (p=0.50). However, DM produced a significantly reduced cholesterol efflux in Hp 2 mice (p=0.0001 comparing efflux in Hp 2 DM mice with Hp 2 mice). The reduction in efflux produced by DM in Hp 2 mice was prevented by administration of vitamin E to these mice (p=0.43 comparing efflux in Hp 2 DM mice with vitamin E and Hp 2 mice).

### DETAILED DESCRIPTION

In one embodiment, provided herein are to methods of determining the prospective benefits of reverse cholesterol transport therapy, and in particular utility of cholesteryl ester transfer protein inhibitors, for prevention or treatment of cardiovascular disease in individuals, based on polymorphism at the haptoglobin 2 allele and subsequent therapies.

Haptoglobin (Hp) is a highly conserved plasma glycoprotein and is the major protein that binds free hemoglobin (Hb) with a high avidity (kd, ˜1×10⁻¹⁵ mol/L). Extracorpuscular hemoglobin (Hb) is rapidly bound by Hp. The role of the Hp-Hb complex in modulating oxidative stress and inflammation associated with cardiovascular disease (CVD) is Hp genotype dependent.

In one embodiment, plasma levels of HDL and its major constituent protein apolipoprotein A-I (apoA-I) are inversely correlated with the incidence of atherosclerotic cardiovascular disease. In another embodiment, HDL and apoA-I confer protection against atherosclerosis by promoting cholesterol efflux from macrophages in a process termed in another embodiment, “reverse cholesterol transport (RCT)”. In one embodiment, the interaction of apoA-I with the ATP-binding cassette protein A1 and the activation of the enzyme lecithin/cholesterol acyltransferase (LCAT) are critical steps in the production of functional HDL and the RCT process. In one embodiment, in subjects having diabetes, the plasma level of HDL is reduced and in another embodiment, the atheroprotective role of the existing HDL is impaired. In one embodiment, the loss of the protective role of HDL in diabetes is due to oxidative modification of apoA-I, which severely impairs cholesterol efflux from macrophages by the ATP-binding cassette protein A1 and LCAT pathways.

In one embodiment, ApoA-I stimulates the efflux of cholesterol from cell toward HDL and the enzyme LCAT to convert, on the HDL surface, cell-derived cholesterol into cholesteryl ester, which is placed in another embodiment into the lipoprotein core and transported through circulation to liver for catabolism and bile production. In one embodiment, factors interacting with ApoA-I interfere with such activity or, in one embodiment, the reverse cholesterol transport.

In one embodiment, ApoA-I binds haptoglobin (Hp) in blood. Two common alleles exist at the haptoglobin (Hp) locus, located on chromosome 16q 13, near the lecithin: cholesterol acyltransferase (LCAT) and the human CETP gene loci and the Hp2 allele is associated with an increased incidence of cardiovascular disease, specifically in diabetes mellitus (DM).

Oxidative stress is increased in Hp2 mice and humans with DM. Oxidative modification of the apolipoprotein A-I inhibits reverse cholesterol transport. Studies were conducted to test the hypothesis that reverse cholesterol transport is impaired in Hp2 DM mice and humans. In vitro, using serum from non-DM and DM individuals, cholesterol efflux was measured from ³H-cholesterol-labeled macrophages. In vivo, ³H-cholesterol-loaded macrophages were injected intraperitoneally into non-DM and DM mice with the Hp1-1 or Hp2-2 genotype and ³H-tracer levels were monitored in plasma, liver, and feces. As will be seen in the Examples below, in diabetes, significantly decreased cholesterol efflux was seen from macrophages incubated with serum from Hp2-1 or Hp2-2 as compared with Hp1-1 individuals (P<0.01). The interaction between Hp type and DM was recapitulated using purified Hp and glycated Hb. In vivo, DM mice loaded with ³H-cholesterol-labeled macrophages had a 40% reduction in ³H-cholesterol in plasma, liver, and feces as compared with non-DM mice (P<0.01). The reduction in reverse cholesterol transport associated with DM was significantly greater in Hp2-2 mice as compared with Hp1-1 mice (54% versus 25% in plasma; 52% versus 27% in liver; 57% versus 32% in feces; P_{—}0.03). Reverse cholesterol transport is thus decreased in Hp2-2 DM, and may explain in part the increased atherosclerotic burden found in Hp2-2 DM individuals.

Haptoglobin is inherited by two co-dominant autosomal alleles situated on chromosome 16 in humans, these are Hpl and Hp2. There are three phenotypes Hp1-1, Hp2-1 and Hp2-2. Haptoglobin molecule is a tetramer comprising of four polypeptide chains, two alpha and two beta chains, of which alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp1-1 individuals have greater hemoglobin binding capacity when compared to those individuals with Hp2-1 and Hp2-2. The gene differentiation to Hp-2 from Hp-1 resulted in a dramatic change in the biophysical and biochemical properties of the haptoglobin protein encoded by each of the 2 alleles.

In another embodiment, Hp inhibits ApoA-I-dependent LCAT activity and in another embodiment, is associated with low reverse cholesterol transport in human ovarian follicular fluid. In another embodiment, free hemoglobin (Hb) competes with ApoA-I for binding Hp, in a site different from that involved in the ApoA-I binding. In one embodiment, a domain, localized in the amino acid sequence spanning from Glu¹¹³ to Asn¹⁸⁴, on ApoA-I binds Hp on a site overlapping with the site responsible for activation of the enzyme lecithin/cholesterol acyltransferase (LCAT). In one embodiment, Hp-2-2 binds ApoA-I with higher affinity than Hp-1-1, thereby reducing activation of LCAT in DM subjects.

Thus, the studies described here support the correlation between Hp genotype and the benefit to a patient of therapy using a cholesteryl ester transfer protein inhibitor.

While these observations were obtained in the study of diabetic animals, similar conclusions can be made for individuals with vascular complications who are not diabetic. This, all of the embodiments herein are applicable to diabetic patients as well as non-diabetic patients.

Accordingly and in one embodiment, provided herein is a method of determining prognosis for a subject having a vascular complication, to benefit from treatment with reverse cholesterol transport therapy comprising the step of obtaining a biological sample from the subject; and determining the subject's haptoglobin allelic genotype, whereby a subject expressing the Hp-2-2 genotype will benefit from treatment with reverse cholesterol transport therapy.

In one embodiment, “benefit from treatment with reverse cholesterol transport therapy” refers to the degree by which plasma HDL is increased due to the inhibition of CETP that transfers cholesterol esters from HDL to lower-density lipoproteins, thereby boosting reverse cholesterol transport.

The embodiments described herein are directed to a method of assessing the benefit to patients of a cholesterol transport therapy, and typically CETP inhibitor therapy, so as to allow for preventive medicine to be practiced where applicable. More particularly, the benefit to the patient is more pronounced in a patient having a haptoglobin 2-2 phenotype compared to patients having haptoglobin 1-2 phenotype or haptoglobin 1-1 phenotypes.

Furthermore, other therapies directed towards enhancing reverse cholesterol transport are fully encompassed within the methods described herein. Such strategies as modifying the activities of lecithin cholesterol acyltransferase (LCAT) in one embodiment, or in other embodiments ATP binding cassette transporter 1 (ABCA1), hepatic lipase (HL), and membrane modulators are also benefited by determining the patient's haptoglobin genotype. Within the CETP class in addition to torcetrapib are such compounds in development including JTT-705 (Japan Tobacco; also called RO 4607381 by Roche), and a CETP vaccine from Avant. These and other compounds enhancing reverse cholesterol transport are fully inclusive of the embodiments herein.

As shown in the examples below, administration of vitamin E to diabetic, Hp 2-2 mice was found to prevent the impairment in efflux produced by DM in Hp 2 mice. This, in another embodiment, vitamin E can be used to improve or correct the defective or impaired reverse cholesterol transport in subjects who are Hp 2-2, including diabetic subjects. In a further embodiment, other antioxidants such as but not limited to glutathione peroxidase and its mimetics can be used to improve or correct the impaired reverse cholesterol transport therein. Non-limiting examples of glutathione peroxidase mimetics are described herein. Moreover, in further embodiments, combinations of antioxidants can be used to achieve a similar effect, and one or more antioxidants can be combined with a cholesterol ester transport protein inhibitor such as torcetrapib to achieve the desired effect.

The present invention also provides a kit for evaluating the potential benefit to a patient to benefit from cholesterol transport therapy for treatment of a vascular complication. The kit comprises packaged reagents for determining a haptoglobin phenotype of the patient and the kit is identified for use in evaluating a potential of a patient to benefit from, for example, CETP inhibitor for treatment of a vascular complication. The nature of these reagents will become apparent to those of skill in the art from the following descriptions and further from well known and characterized sequence data of the haptoglobin 1 and 2 alleles.

In one embodiment, atherosclerotic disease manifests as cardiovascular diseases such as coronary artery disease. In another embodiment, the cardiovascular disease is angina. In another embodiment, the cardiovascular disease is MI. In another embodiment, the vascular disease is cerebrovascular diseases such as stroke that are typical sequelae of lipid abnormalities. In addition to these complications and in another embodiment, those that diabetics are at risk of developing, including diabetic retinopathy, diabetic cataracts and glaucoma, diabetic nephropathy, diabetic neuropathy, claudication, and gangrene.

In a further embodiment, the vascular complication is a macrovascular complication In another embodiment, the vascular disease is a chronic heart failure. In another embodiment, the vascular disease is cardiovascular death. In another embodiment, the vascular disease is stroke. In another embodiment, the vascular disease is myocardial infarction. In another embodiment, the vascular disease is coronary angioplasty associated restenosis. In another embodiment, the vascular disease is fewer coronary artery collateral blood vessels and myocardial ischemia in other embodiments. In another embodiment, the vascular complication is a microvascular complication, such as diabetic neuropathy in one embodiment. In another embodiment, the microvascular disease is diabetic nephropathy or diabetic retinopathy in yet another embodiment.

Microvascular disease may be characterized in one embodiment, by an unevenly distributed thickening (or hyalinization) of the intima of small arterioles, due in another embodiment, to the accumulation of type IV collagen in the basement membrane, or microaneurysm of the arterioles, which compromises the extent of the maximal arteriolar dilation that can be achieved and impairs the delivery of nutrients and hormones to the tissues, or to remove waste in another embodiment. The vasculature distal to the arterioles may also be affected in one embodiment, such as by increased capillary basement membrane thickening, abnormalities in endothelial metabolism, or via impaired fibrinolysis, also resulting in reduced delivery of nutrients and hormones to the tissues, or waste removal in another embodiment.

Lipoproteins have in one embodiment, the function of transporting lipids throughout the body. Low density lipoproteins are responsible in another embodiment, for the transport of cholesterol with the protein moiety involved: apolipoprotein (Apo) B. Very low density lipoproteins are responsible in one embodiment, for the transport of triglycerides with the protein moiety involved: Apo E. In another embodiment, HDLs are responsible for reverse cholesterol transport and in one embodiment, play an important role in being a naturally occurring potent anti-inflammatory and antioxidant agent with the protein moiety involved: Apo A. It is the protein moiety of the lipoproteins that is modified in one embodiment, by the processes of oxidation, glycation, and glycoxidation with a resultant increase in redox stress and the production of ROS. In one embodiment, the modification of the protein moiety is responsible for their retention within the intima, inducing in one embodiment, atherogenesis and thus atheroscleropathy. Accordingly and in one embodiment, Hp genotype is predictive of the extent of glycoxidation capable of modifying Apo A, thereby leading to increased redox stress, wherein the extent of glycoxidation or in one embodiment, oxidation, decreases from Hp-2-2, to Hp-2-1, to Hp-1-1, and is diagnosed according to the methods provided herein.

In one embodiment, reverse cholesterol transport (RCT), is influenced in large part by the quantity and quality of HDL. In another embodiment, the HDL-mediated cholesterol efflux being elicited with serum from DM individuals with the different Hp types is markedly different. In one embodiment, Hp-genotype dependence of cholesterol efflux reflects differences in LCAT cholesterol esterification rate. In one embodiment Hp binds to apolipoprotein A-1, which overlaps with the binding site of LCAT. In another embodiment, displacement of LCAT from apolipoprotein A-1 results in an inhibition of LCAT cholesterol esterification rate and in a reduction of RCT in human ovarian follicular fluid in another embodiment. In one embodiment, decoy peptides corresponding to the Leu141-Ala169 region of apolipoprotein A-1 block the ability of Hp to reduce LCAT cholesterol esterification rate.

In one embodiment, oxidative mechanisms impair the ATP-binding cassette protein A1 transporter, a component of the RCT process. In another embodiment, a primary culprit oxidant mediating Hp-type dependent differences in oxidative stress has been identified as non-transferrin-bound iron.

Several functions have been assigned to the haptoglobin protein that may impact on the development of atherosclerosis. It has been appreciated for over 60 years that a major function of serum haptoglobin is to bind free hemoglobin. This interaction is thought to help scavenge iron and prevent its loss in the urine and to serve as an antioxidant thereby protecting tissues against hemoglobin mediated tissue oxidation. The antioxidant capacity of the different haptoglobin phenotypes has been shown to differ with the haptoglobin 1-1 protein appearing to confer superior antioxidant protection as compared to the other forms of the protein. Such an antioxidant hypothesis is particularly intriguing given the apparent important role of oxidative stress in the development of diabetic vascular complications. Perhaps further amplifying apparent differences in the oxidative protection afforded by the different types of haptoglobin are gross differences in size of the haptoglobin protein present in individuals with the different phenotypes. Haptoglobin 1-1 is markedly smaller then haptoglobin 2-2 and thus may be better able to sieve into the extravascular compartment and prevent hemoglobin mediated tissue damage at sites of vascular injury.

Also embodied herein are findings related to the clearance from circulation of complexes between hemoglobin and the various haptoglobin phenotypes, and their relationship to enhancing oxidative stress in patients dependent on haptoglobin phenotype. In a further embodiment, in Hp 2 mice the Hp-Hb complex is cleared much more slowly than in Hp 1 mice. In another embodiment, the Hp-Hb complex is cleared particularly slow in the setting of DM, resulting in a higher steady state plasma concentration of Hp-Hb in Hp 2 DM mice. As mentioned above, haptoglobin (Hp) is a highly conserved plasma glycoprotein and is the major protein that binds free hemoglobin (Hb) with a high avidity (kd, ˜1×10⁻¹⁵ mol/L). Extracorpuscular hemoglobin (Hb) is rapidly bound by Hp. In one embodiment, the role of the Hp-Hb complex in modulating oxidative stress and inflammation associated with cardiovascular disease (CVD) is Hp genotype dependent.

In another embodiment, Hp-Hb complex binds to HDL and Hp and DM dependent differences in the clearance of the complex result in another embodiment, in a greater fraction of HDL being modified by Hp-Hb complex in Hp 2 DM individuals. In one embodiment, the impairment in reverse cholesterol transport in Hp 2 DM individuals is due to increased oxidative modification by Hp 2-Hb complex bound to HDL. In another embodiment, the Hp-Hb complex in subjects expressing Hp-2-2 allele is cleared much more slowly than in subjects expressing Hp-2-2 allele, particularly and in another embodiment, in the setting of DM, resulting in a higher steady state plasma concentration of Hp-Hb. In another embodiment, a significant fraction of plasma Hp-Hb binds to HDL and since there is more Hp-Hb complex in subjects expressing Hp-2-2 allele, a significantly higher fraction of HDL in subjects expressing Hp-2-2 allele will have Hp-Hb bound to it, thereby affecting the ability of HDL to carry out reverse cholesterol transport.

In another embodiment, vitamin E dramatically reduces cardiovascular events when administered to Hp 2 DM individuals. In Hp transgenic mice, antioxidant therapy provides in another embodiment, cardiovascular benefit only to Hp 2 mice but not to Hp 1 DM mice. In one embodiment transgenic mice expressing human Hp-2-2 may be used as a platform on which to rationally design antioxidant and other adjunctive therapies to improve HDL function in Hp 2 DM individuals and thereby reduce the enormous burden of CVD in this cohort is carried out.

Lipoproteins have in one embodiment, the function of transporting lipids throughout the body. Low density lipoproteins are responsible in another embodiment, for the transport of cholesterol with the protein moiety involved: apolipoprotein (Apo) B. Very low density lipoproteins are responsible in one embodiment, for the transport of triglycerides with the protein moiety involved: Apo E. In another embodiment, HDLs are responsible for reverse cholesterol transport and in one embodiment, play an important role in being a naturally occurring potent anti-inflammatory and antioxidant agent with the protein moiety involved: Apo A. It is the protein moiety of the lipoproteins that is modified in one embodiment, by the processes of oxidation, glycation, and glycoxidation with a resultant increase in redox stress and the production of ROS. In one embodiment, the modification of the protein moiety is responsible for their retention within the intima, inducing in one embodiment, atherogenesis and thus atheroscleropathy. Accordingly and in one embodiment, Hp genotype is predictive of the extent of glycoxidation capable of modifying Apo A, thereby leading to increased redox stress, wherein the extent of glycoxidation or in one embodiment, oxidation, decreases from Hp-2-2, to Hp-2-1, to Hp-1-1, and is diagnosed according to the methods provided herein.

In one embodiment, antioxidant therapy may be beneficial in specific subgroups with increased oxidative stress. Oxidative Stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues such as glial cells and neurons, must be able to protect themselves against the toxic effects of ROS. In one embodiment ROS-detoxifying enzymes have an important role in epithelial wound repair. In another embodiment, the glutathione peroxidase mimetics provided in the compositions and compounds provided herein, replace the ROS detoxifying enzymes described herein.

In one embodiment, overproduction of reactive oxygen species (ROS) including hydrogen peroxide (H₂O₂), superoxide anion (O^{.}₂⁻); nitric oxide (NO^{.}) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPX) and catalase are, involved in the disposal of O^{.}₂⁻ and H₂O₂. First, SOD catalyses the dismutation of O^{.}₂⁻ to H₂O₂ and molecular oxygen (O₂), resulting in selective O^{.}₂⁻ scavenging. Then, GPX and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When free radicals are generated in excess or when FRSEs are defective, H₂O₂ is reduced into hydroxyl radical (OH^{.}), which is one of the highly reactive ROS responsible in one embodiment for initiation of lipid peroxidation of cellular membranes. In another embodiment, organic peroxide-induced lipid peroxidation is implicated as one of the essential mechanisms of toxicity in the death of hippocampal neurons. In one embodiment, an indicator of the oxidative stress in the cell is the level of lipid peroxidation and its final product is MDA. In another embodiment the level of lipid peroxidation increases in inflammatory diseases, such as meningitis in one embodiment. In one embodiment, the compounds provided herein and in another embodiment, are represented by the compounds of formula I-X, are effective antioxidants, capable of reducing lipid peroxidation, or in another embodiment, are effective as anti-inflammatory agents.

In one embodiment, reverse cholesterol transport (RCT), is influenced in large part by the quantity and quality of HDL. In another embodiment, the HDL-mediated cholesterol efflux being elicited with serum from DM individuals with the different Hp types is markedly different. In one embodiment, Hp-genotype dependence of cholesterol efflux reflects differences in LCAT cholesterol esterification rate.

According to various typical embodiments of the method of the present invention, determining the haptoglobin phenotype of a subject is effected by any one of a variety of methods including, but not limited to, a signal amplification method, a direct detection method and detection of at least one sequence change. These methods determine a phenotype indirectly, by determining a genotype. As will be explained hereinbelow, determination of a haptoglobin phenotype may also be accomplished directly by analysis of haptoglobin gene products.

The signal amplification method according to various preferred embodiments of the present invention may amplify, for example, a DNA molecule or an RNA molecule. Signal amplification methods which might be used as part of the present invention include, but are not limited to PCR, LCR (LAR), Self-Sustained Synthetic Reaction (3SR/NASBA) or a Q-Beta (Q.beta.) Replicase reaction.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] described by Barany, Proc. Natl. Acad. Sci., 88:189 (1991); Barany, PCR Methods and Applic., 1:5 (1991); and Wu and Wallace, Genomics 4:560 (1989) has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes. Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR1NASBA): The self-sustained sequence replication reaction (3SR) (Guatelli et al., Proc. Natl. Acad. Sci., 87:1874-1878, 1990), with an erratum at Proc. Natl. Acad. Sci., 87:7797, 1990) is a transcription-based in vitro amplification system (Kwok et al., Proc. Natl. Acad. Sci., 86:1173-1177, 1989) that can exponentially amplify RNA sequences at a uniform temperature. The amplified RNA can then be utilized for mutation detection (Fahy et al., PCR Meth. Appl., 1:25-33, 1991). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Q.beta.) Replicase: In this method, a probe which recognizes the sequence of interest is attached to the replicatable RNA template for Q.beta. replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Q.beta. systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res., 18:999, 1990).

A similar 3′-mismatch strategy is used with greater effect to prevent ligation in the LCR (Barany, PCR Meth. Applic., 1:5, 1991). Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), described by Urdea et al., Gene 61:253-264 (1987), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perhnan and Butow, Science 246:1106, 1989), but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Determination of a haptoglobin phenotype may, as if further exemplified in the Examples section that follows, also be accomplished directly, by analyzing the protein gene products of the haptoglobin gene, or portions thereof. Such a direct analysis is often accomplished using an immunological detection method.

Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two haptoglobin alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, haptoglobin in this case and in the methods detailed hereinbelow, with a specific antibody and radiolabelled antibody binding protein (e.g., protein A labeled with I.sup.125) immobilized on a precipitable carrier such as agarose beads. The number of counts in the precipitated pellet is proportional to the amount of substrate.

In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Biologically active derivatives or analogs of the proteins described herein include in one embodiment peptide mimetics. These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis. Accordingly and in another embodiment, the methods described herein are used to identify peptides capable of acting as reverse cholesterol transfer inhibitors.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the haptoglobin phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc.

In one embodiment, the methods described hereinabove are used as part of the methods of treating vascular complications by contacting the subject with compositions comprising cholesteryl ester transfer protein inhibitor and glutathione peroxidase or its isomer, metabolite, and/or salt therefore.

Accordingly and in another embodiment, provided herein is a method of treating, or in another embodiment inhibiting or suppressing, or in another embodiment reducing symptoms associated with a vascular complication in a subject comprising the step of contacting the subject with an effective amount of a composition comprising glutathione peroxidase or its isomer, metabolite, and/or salt therefore, and cholesteryl ester transfer protein inhibitor thereby reducing symptoms associated with vascular complication. In another embodiment, the glutathione peroxidase or its mimetic, isomer, metabolite, and/or salt therefore is represented by the compounds of formula I-X.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

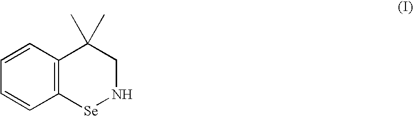

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of 7 electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet. In vitro and in vivo studies with the compound of formula 1, a show in one embodiment, that glutahione peroxidase or its mimetic, isomer, metabolite, and/or salt therefore is capable of protecting cells against reactive oxygen species.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of GPx, a Gpx mimetic, or its pharmaceutically acceptable salt, its functional derivative, its synthetic analog or a combination thereof, is used in the methods and compositions of the invention.

In one embodiment, the glutathione peroxidase mimetic is represented by formula I:

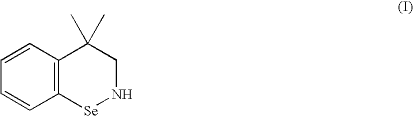

In one embodiment, the compound of formula (II), refers to benzisoselen-azoline or -azine derivatives represented by the following general formula:

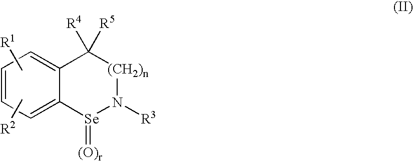

where: R¹, R²=hydrogen; lower alkyl; OR⁶; —(CH₂)ₘ NR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂NR⁶R⁷; —CO(CH₂)ₚCOR⁸; R⁹; R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸; R⁴=lower alkyl; aralkyl; substituted aralkyl; —(CH₂)ₚCOR⁸; —(CH₂)ₚR⁸; F; R⁵=lower alkyl; aralkyl; substituted aralkyl; R⁶=lower alkyl; aralkyl; substituted aralkyl; —(CH₂)ₘCOR⁸; —(CH₂)_{q}R⁸; R⁷=lower alkyl; aralkyl; substituted aralkyl; —(CH₂)ₘCOR⁸; R⁸=lower alkyl; aralkyl; substituted aralkyl; aryl; substituted aryl; heteroaryl; substituted heteroaryl; hydroxy; lower alkoxy; R⁹; R⁹=

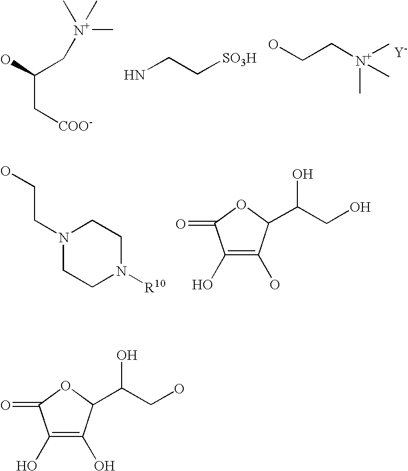

R¹⁰=hydrogen; lower alkyl; aralkyl or substituted aralkyl; aryl or substituted aryl; Y⁻ represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl) or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety.

In one embodiment, the glutathione peroxidase or its mimetic, isomer, metabolite, and/or salt therefore, used in the methods and compositions provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating a vascular complication in a subject comprising the step of contacting the subject with an effective amount of a composition comprising an organoselenium compound, and cholesteryl ester transfer protein inhibitor thereby inhibiting or suppressing vascular complication and increasing endogenous anti-oxidant ability of the cells, or in another embodiment, scavenging free radicals causing apoptosis of vascular cells and their associated pathologies.

Accordingly and in another embodiment, provided herein is a composition for treating a vascular complication in a subject comprising: a therapeutically effective amount of a composition comprising glutathione peroxidase or its isomer, metabolite, and/or salt therefore and cholesteryl ester transfer protein inhibitor. In another embodiment, the compositions described herein, are used in the methods provided herein.

In another embodiment, the glutathione peroxidase or its mimetic, isomer, metabolite, and/or salt therefore used in the compositions and methods provided herein, is represented by the compound of formula III:

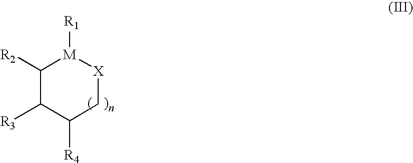

wherein,
the compound of formula III is a ring; and

### X is O or NH

### M is Se or Te

n is 0-2
R₁ is oxygen; and
forms an oxo complex with M; or
R₁ is oxygen or NH; and
forms together with the metal, a 4-7 member ring, which optionally is substituted by an oxo group; or forms together with the metal, a first 4-7 member ring, which is optionally substituted by an oxo group, wherein said first ring is fused with a second 4-7 member ring, wherein said second 4-7 member ring is optionally substituted by alkyl, alkoxy, nitro, aryl, cyano, amino, halogen, or —NH(C═O)R or —SO₂R where R is alkyl or aryl;
R₂, R₃ and R₄ are independently hydrogen, alkyl, oxo, amino or together with the organometalic ring to which two of the substituents are attached, a fused 4-7 member ring system wherein said 4-7 member ring is optionally substituted by alkyl, alkoxy, nitro, aryl, cyano, amino, halogen, or —NH(C═O)R or —SO₂R where R is alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometalic ring a fused benzene ring, R₄ is hydrogen, then M is Se; or
if R₄ is an oxo group, and R₂ and R₃ form together with the organometalic ring a fused benzene ring, R₁ is oxygen, n is 0 and forms together with the metal a first 5 member ring, substituted by an oxo group α to R₁, and said ring is fused to a second benzene ring, then M is Te.

In one embodiment, a 4-7 member ring group refers to a saturated cyclic ring. In another embodiment the 4-7 member ring group refers to an unsaturated cyclic ring. In another embodiment the 4-7 member ring group refers to a heterocyclic unsaturated cyclic ring. In another embodiment the 4-7 member ring group refers to a heterocyclic saturated cyclic ring. In one embodiment the 4-7 member ring is unsubstituted. In one embodiment, the ring is substituted by one or more of the following: alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl.

In one embodiment, substituent groups may be attached via single or double bonds, as appropriate, as will be appreciated by one skilled in the art.

According to embodiments herein, the term alkyl as used throughout the specification and claims may include both “unsubstituted alkyls” and/or “substituted alkyls”, the latter of which may refer to alkyl moieties having substituents replacing hydrogen on one or more carbons of the hydrocarbon backbone. In another embodiment, such substituents may include, for example, a halogen, a hydroxyl, an alkoxyl, a silyloxy, a carbonyl, and ester, a phosphoryl, an amine, an amide, an imine, a thiol, a thioether, a thioester, a sulfonyl, an amino, a nitro, or an organometallic moiety. It will be understood by those skilled in the art that the moieties substituted on the hydrocarbon chain may themselves be substituted, if appropriate. For instance, the substituents of a substituted alkyl may include substituted and unsubstituted forms of amines, imines, amides, phosphoryls (including phosphonates and phosphines), sulfonyls (including sulfates and sulfonates), and silyl groups, as well as ethers, thioethers, selenoethers, carbonyls (including ketones, aldehydes, carboxylates, and esters), —CF₃, and —CN. Of course other substituents may be applied. In another embodiment, cycloalkyls may be further substituted with alkyls, alkenyls, alkoxys, thioalkyls, aminoalkyls, carbonyl-substituted alkyls, CF₃, and CN. Of course other substituents may be applied.

In another embodiment, a compound of formula IV is provided, wherein M, R₁ and R₄ are as described above for formula III.

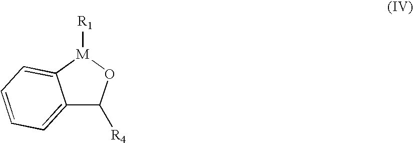

In another embodiment, a compound of formula V is provided, wherein M, R₂, R₃ and R₄ are as described above for formula III.

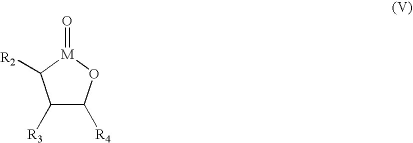

In another embodiment, a compound of formula VI is provided, wherein M, R₂, R₃ and R₄ are as described above for formula III.

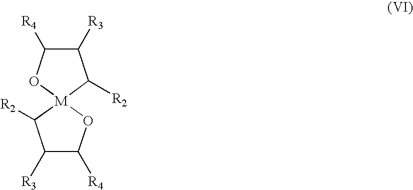

In another embodiment, a compound of formula (VII) is provided, wherein M, R₂ and R₃ are as described above for formula III

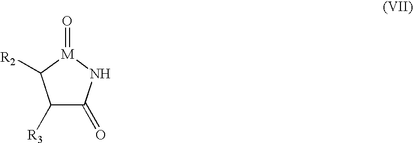

In another embodiment, a compound of formula VIII is provided, wherein M, R₂ and R₃ are as described above for formula III.

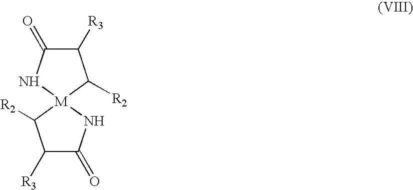

In one embodiment, the compound of formula III, used in the compositions and methods provided herein, is represented by any one of the following compounds or their combinations:

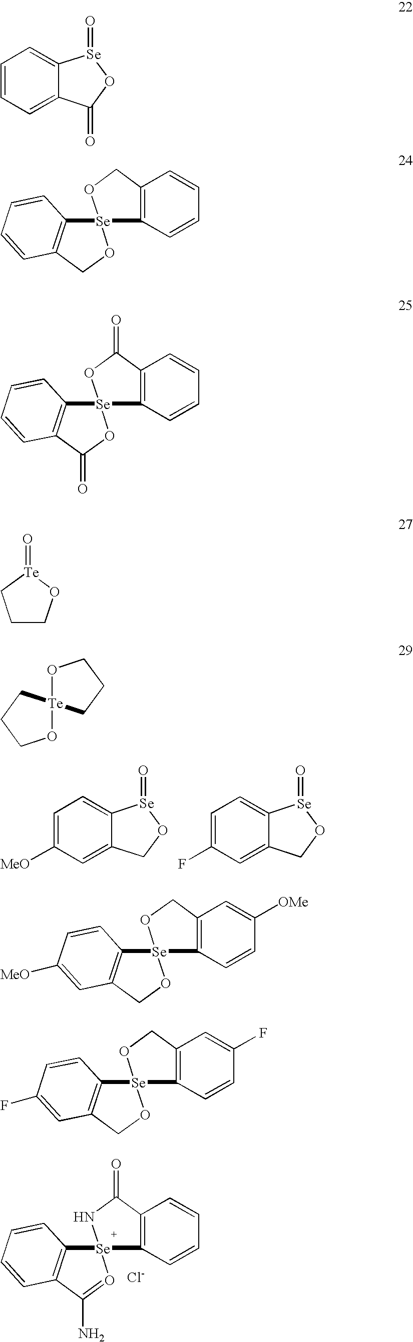

In another embodiment, the glutathione peroxidase or its isomer, metabolite, and/or salt therefore used in the compositions and methods provided herein, is represented by the compound of formula IX:

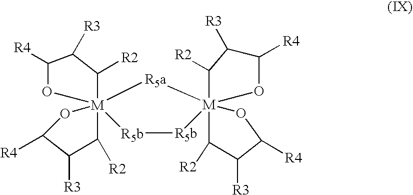

wherein,

### M is Se or Te;

R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 membered ring system, wherein said 4-7 membered ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and

R₅ₐ or R_{5b} is one or more oxygen, carbon, or nitrogen atoms and forms a neutral complex with the chalcogen.

In one embodiment, the compound represented formula (IX), is represented by the compound of formula X:

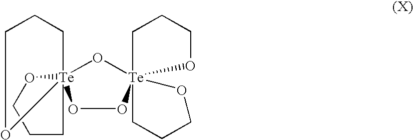

In one embodiment, the compounds represented by formula I-X, mimic the in-vivo activity of glutathione peroxidase. The term “mimic” refers, in one embodiment to comparable, identical, or superior activity, in the context of conversion, timing, stability or overall performance of the compound, or any combination thereof.

Biologically active derivatives or analogs of the proteins described herein include in one embodiment peptide mimetics. These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the compositions provided herein are used for the treatment of vasospasm conditions and may be present in the form of suspension or dispersion form in solvents or fats, in the form of a nonionic vesicle dispersion or else in the form of an emulsion, preferably an oil-in-water emulsion, such as a cream or milk, or in the form of an ointment, gel, cream gel, sun oil, solid stick, powder, aerosol, foam or spray.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In some embodiments, the compositions and methods provided herein permit direct application to the site where it is needed. In the practice of the methods provided herein, it is contemplated that virtually any of the compositions provided herein can be employed.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In another embodiment, the compositions provided herein are suitable for oral, intraoral, rectal, parenteral, topical, epicutaneous, transdermal, subcutaneous, intramuscular, intranasal, sublingual, buccal, intradural, intraocular, intrarespiratory, nasal inhalation or a combination thereof. In one embodiment, the step of administering the compositions provided herein, in the methods provided herein is carried out as oral administration, or in another embodiment, the administration of the compositions provided herein is intraoral, or in another embodiment, the administration of the compositions provided herein is rectal, or in another embodiment, the administration of the compositions provided herein is parenteral, or in another embodiment, the administration of the compositions provided herein is topical, or in another embodiment, the administration of the compositions provided herein is epicutaneous, or in another embodiment, the administration of the compositions provided herein is transdermal, or in another embodiment, the administration of the compositions provided herein is subcutaneous, or in another embodiment, the administration of the compositions provided herein is intramuscular, or in another embodiment, the administration of the compositions provided herein is intranasal, or in another embodiment, the administration of the compositions provided herein is sublingual, or in another embodiment, the administration of the compositions provided herein is buccal, or in another embodiment, the administration of the compositions provided herein is intradural, or in another embodiment, the administration of the compositions provided herein is intraocular, or in another embodiment, the administration of the compositions provided herein is intrarespiratory, or in another embodiment, the administration of the compositions provided herein is nasal inhalation or in another embodiment, the administration of the compositions provided herein is a combination thereof.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I-X are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations comprising the compositions used in one embodiment in the methods provided herein, can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition described in the embodiments provided herein, can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the compositions described herein, which are used in another embodiment, in the methods provided herein, further comprise a carrier, an excipient, a lubricant, a flow aid, a processing aid or a diluent.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. EngI. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the methods provided herein comprise contacting the subject with the compositions described herein. Accordingly and in one embodiment provided herein is a method of treating a subject having a vascular complication, wherein the vascular complication is hypercholesterolemia, comprising the step of contacting the subject with an effective amount of a composition comprising glutathione peroxidase or its isomer, metabolite, and/or salt therefore, represented by any one of the compounds of formula I-X or their combination, and cholesteryl ester transfer protein inhibitor, such as torcetrapib in one embodiment, thereby treating vascular complication.

In another embodiment, the vascular complication sought to be treated using the methods and compositions described herein, is cardiovascular complication that is angina in one embodiment. In another embodiment the cardiovascular complication is myocardial infarct. In another embodiment the cardiovascular complication is peripheral vascular disease. In another embodiment the cardiovascular complication is cerebrovascular disease, or in another embodiment the cardiovascular complication is a combination thereof.

In one embodiment, the methods provided herein, using the compositions provided herein, further comprise contacting the subject with one or more additional agent. In another embodiment, the additional agent which is not glutathione peroxidase or its isomer, metabolite, and/or salt therefore, nor cholesteryl ester transfer protein inhibitor, is an aldosterone inhibitor. In another embodiment, the additional agent is an angiotensin-converting enzyme. In another embodiment, the additional agent is an antioxidant. In another embodiment, the additional agent is an angiotensin receptor AT₁ blocker (ARB). In another embodiment, the additional agent is an angiotensin II receptor antagonist. In another embodiment, the additional agent is a calcium channel blocker. In another embodiment, the additional agent is a diuretic. In another embodiment, the additional agent is digitalis.

In another embodiment, the additional agent is a beta blocker. In another embodiment, the additional agent is a statin. In another embodiment, the additional agent is a cholestyramine or in another embodiment, the additional agent is a combination thereof.

In one embodiment, the additional therapeutic agent used in the methods and compositions described herein is a statin. In another embodiment, the term “statins” refers to a family of compounds that are inhibitors of 3-hydroxy-3-methylglutaryl coenzyme A (HMG-CoA) reductase, the rate-limiting enzyme in cholesterol biosynthesis. As HMG-CoA reductase inhibitors, in one embodiment, statins reduce plasma cholesterol levels in various mammalian species.

Statins inhibit in one embodiment, cholesterol biosynthesis in humans by competitively inhibiting the 3-hydroxy-3-methyl-glutaryl-coenzyme A (“HMG-CoA”) reductase enzyme. HMG-CoA reductase catalyzes in another embodiment, the conversion of HMG to mevalonate, which is the rate determining step in the biosynthesis of cholesterol. Decreased production of cholesterol causes in one embodiment, an increase in the number of LDL receptors and corresponding reduction in the concentration of LDL particles in the bloodstream. Reduction in the LDL level in the bloodstream reduces the risk of coronary artery disease.

In one embodiment, the statins used in the compositions and methods of the invention are lovastatin (referred to as mevinolin in one embodiment, or monacolin-K in another embodiment), or compactin (referred to as mevastatin in one embodiment, or ML-236B in another embodiment), pravastatin, atorvastatin (Lipitor) rosuvastatin (Crestor) fluvastatin (Lescol), simvastatin (Zocor), cerivastatin or their combination in other embodiments. In one embodiment, the statin used as one or more additional therapeutic agent, is any one of the statins described herein, or in another embodiment, in combination of statins. A person skilled in the art would readily recognize that the choice of statin used, will depend on several factors, such as in certain embodiment, the underlying condition of the subject, other drugs administered, other pathologies and the like.

The renin-angiotensin-aldosterone system (“RAAS”) is involved in one embodiment, in regulating pressure homeostasis and also in the development of hypertension, a condition shown as a major factor in the progression of cardiovascular diseases. Secretion of the enzyme renin from the juxtaglomerular cells in the kidney activates in another embodiment, the renin-angiotensin-aldosterone system (RAAS), acting on a naturally-occurring substrate, angiotensinogen, to release in another embodiment, a decapeptide, Angiotensin I. Angiotensin converting enzyme (“ACE”) cleaves in one embodiment, the secreted decapeptide, producing an octapeptide, Angiotensin II, which is in another embodiment, the primary active species of the RAAS system. Angiotensin II stimulates in one embodiment, aldosterone secretion, promoting sodium and fluid retention, inhibiting renin secretion, increasing sympathetic nervous system activity, stimulating vasopressin secretion, causing a positive cardiac inotropic effect or modulating other hormonal systems in other embodiments.

In one embodiment, the angiotensin converting enzyme (ACE) inhibitor used in the methods and compositions of the invention is captopril. In another embodiment, the ACE inhibitor is cilazapril. In another embodiment, the ACE inhibitor is delapril. In another embodiment, the ACE inhibitor is enalapril. In another embodiment, the ACE inhibitor is fentiapril. In another embodiment, the ACE inhibitor is fosinopril. In another embodiment, the ACE inhibitor is indolapril. In another embodiment, the ACE inhibitor is lisinopril. In another embodiment, the ACE inhibitor is perindopril. In another embodiment, the ACE inhibitor is pivopril. In another embodiment, the ACE inhibitor is quinapril. In another embodiment, the ACE inhibitor is ramipril. In another embodiment, the ACE inhibitor is spirapril. In another embodiment, the ACE inhibitor is trandolapril. In another embodiment, the ACE inhibitor is zofenopril or a combination thereof in other embodiments.

A representative group of ACE inhibitors that may be used in the compositions and methods provided herein, consists in another embodiment, of the following compounds: AB-103, ancovenin, benazeprilat, BRL-36378, BW-A575C, CGS-13928C, CL-242817, CV-5975, Equaten, EU-4865, EU-4867, EU-5476, foroxymithine, FPL 66564, FR-900456, Hoe-065, 15B2, indolapril, ketomethylureas, KRI-1177, KRI-1230, L-681176, libenzapril, MCD, MDL-27088, MDL-27467A, moveltipril, MS-41, nicotianamine, pentopril, phenacein, pivopril, rentiapril, RG-5975, RG-6134, RG-6207, RGH-0399, ROO-911, RS-10085-197, RS-2039, RS 5139, RS 86127, RU-44403, S-8308, SA-291, spiraprilat, SQ-26900, SQ-28084, SQ-28370, SQ-23940, SQ-31440, Synecor, utibapril, WF-10129, Wy-44221, Wy-44655, Y-23785, Yissum P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, 1-(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-6564, idrapril, Marion Merrell Dow MDL-100240, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, saralasin acetate, temocapril, trandolapril, ceranapril, moexipril, quinaprilat and spirapril.

In one embodiment, the terms “aldosterone antagonist” and “aldosterone receptor antagonist” refer to a compound that inhibits the binding of aldosterone to mineralocorticoid receptors, thereby blocking the biological effects of aldosterone. In one embodiment, the term “antagonist” in the context of describing compounds according to the invention refers to a compound that directly or in another embodiment, indirectly inhibits, or in another embodiment suppresses aldosterone activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the aldosterone, even when there are spare (unbound) aldosterone present. In another embodiment, the term “partial antagonist” refers to a compound does not evoke the maximal inhibitory response from the androgen receptor, even when present at concentrations sufficient to saturate the androgen receptors present.

The aldosterone antagonists used in the methods and compositions of the present invention are in one embodiment, spirolactone-type steroidal compounds. In another embodiment, the term “spirolactone-type” refers to a structure comprising a lactone moiety attached to a steroid nucleus, such as, in one embodiment, at the steroid “D” ring, through a spiro bond configuration. A subclass of spirolactone-type aldosterone antagonist compounds consists in another embodiment, of epoxy-steroidal aldosterone antagonist compounds such as eplerenone. In one embodiment, spirolactone-type antagonist compounds consists of non-epoxy-steroidal aldosterone antagonist compounds such as spironolactone. In one embodiment, the invention provides a composition comprising an aldosterone antagonist, its isomer, functional derivative, synthetic analog, pharmaceutically acceptable salt or combination thereof; and a glutathione peroxidase or its isomer, functional derivative, synthetic analog, pharmaceutically acceptable salt or combination thereof, wherein the aldosterone antagonist is epoxymexrenone, or eplerenone, dihydrospirorenone, 2,2; 6,6-diethlylene-3oxo-17alpha-pregn-4-ene-21,17-carbolactone, spironolactone, 18-deoxy aldosterone, 1,2-dehydro-18-deoxyaldosterone, RU28318 or a combination thereof in other embodiments.

In one embodiment, the antioxidants include small-molecule antioxidants and antioxidant enzymes. Suitable small-molecule antioxidants include, in another embodiment, hydralazine compounds, glutathione, vitamin C, vitamin E, cysteine, N-acetyl-cysteine, beta.-carotene, ebselen, ubiquinone, ubiquinol-10, tocopherols, coenzyme Q, and the like. Suitable antioxidant enzymes include in one embodiment superoxide dismutase, catalase, glutathione peroxidase, or a combination thereof. Suitable antioxidants are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996.

As noted herein, vitamin E, among other antioxidants, has been shown to correct the impaired reverse cholesterol transport in diabetic subjects with the Hp 2-2 phenotype. This, vitamin E or other antioxidants can be administered to subjects to treat or correct an impaired reverse cholesterol transport. In one embodiment, vitamin E is added to foods in one of its more chemically stable forms, e.g., .alpha.-tocopherol acetate (also known as .alpha.-tocopheryl acetate). Four different forms of vitamin E (the alcohol and ester forms of synthetic racemic (rac) vitamin E and the alcohol and ester forms of natural (RRR) vitamin E) are commercially available, and because of their differences in bioactivities and molecular weights, are assigned different values of specific activity (IU per milligram) according to the National Formulary as follows: 1 mg all-rac-.alpha.-tocopherol acetate=1.00 IU 1 mg all-rac-.alpha-tocopherol=1.10 IU 1 mg RRR-.alpha-tocopherol acetate=1.36 IU 1 mg RRR-.alpha-tocopherol=1.49 IU.

In one embodiment, the vitamin E is selected from the group consisting of alpha, beta, gamma and delta tocopherols, alpha, beta, gamma and delta tocotrienols, and combinations thereof. In another embodiment, the alpha tocopherol group is selected from the group consisting of synthetic (all-rac) and natural (RRR) alpha-tocopherols, alpha-tocopheryl acetates, and alpha-tocopheryl succinates.

Oxidative stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In one embodiment, overproduction of reactive oxygen species (ROS) including hydrogen peroxide (H₂O₂), superoxide anion (O^{.}₂⁻); nitric oxide (NO^{.}) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPx) and catalase are, involved in the disposal of O^{.}₂⁻ and H₂O₂. First, SOD catalyses the dismutation of O^{.}₂⁻ to H₂O₂ and molecular oxygen (O₂), resulting in selective O^{.}₂⁻ scavenging. Then, GPx and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When in another embodiment, free radicals are generated in excess or when FRSEs are defective, H₂O₂ is reduced into hydroxyl radical (OH^{.}), which is one of the highly reactive ROS responsible in one embodiment for initiation of lipid peroxidation of cellular membranes. In another embodiment, organic peroxide-induced lipid peroxidation is implicated as one of the essential mechanisms of toxicity in keratinocytes. In one embodiment, benzoyl peroxide, a topical agent, shows the ability to induce an inflammatory reaction mediated by oxidative stress in addition to its antibacterial activity, thereby increasing lipid peroxidation. In one embodiment, an indicator of the oxidative stress in the cell is the level of lipid peroxidation and its final product is MDA. In another embodiment the level of lipid peroxidation increases in inflammatory diseases. In one embodiment, the compounds provided herein and in another embodiment, are represented by the compounds of formulas I-XII herein, are effective antioxidants.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In addition to a direct action on arteries and arterioles, angiotensin II (All), is one of the most potent endogenous vasoconstrictors known, exerts in one embodiment, stimulation on the release of aldosterone from the adrenal cortex. Therefore, the renin-angiotensin system, (RAAS) by virtue of its participation in the control of renal sodium handling, plays an important role in cardiovascular hemeostasis.

In another embodiment, the angiotensin II receptor antagonist used in the compositions and methods of the invention is losartan, irbesartan, eprosartan, candesartan, valsartan, telmisartan, zolasartin, tasosartan or a combination thereof. Examples of angiotensin II receptor antagonists used in the compositions and methods of the invention are in one embodiment biphenyltetrazole compounds or biphenylcarboxylic acid compounds or CS-866, losartan, candesartan, valsartan or irbesartan in other embodiments. In one embodiment, where the above-mentioned compounds have asymmetric carbons, the angiotensin II receptor antagonists of the compositions and methods used in the present invention are optical isomers and mixtures of said isomers. In one embodiment, hydrates of the above-mentioned compounds are also included.

In one embodiment, Cyclic fluxes of Ca²⁺ between three compartments—cytoplasm, sarcoplasmic reticulum (SR), and sarcomere—account for excitation-contraction coupling. Depolarization triggers in another embodiment, entry of small amounts of Ca²⁺ through the L-type Ca²⁺ channels located on the cell membrane, which in one embodiment, prompts SR Ca²⁺ release by cardiac ryanodine receptors (RyR's), a process termed calcium-induced Ca²⁺ release. A rapid rise in cytosolic levels results in one embodiment, fostering Ca²⁺-troponin-C interactions and triggering sarcomere contraction. In another embodiment, activation of the ATP-dependent calcium pump (SERCA) recycles cytosolic Ca²⁺ into the SR to restore sarcomere relaxation. In another embodiment, Ca²⁺ channel blockers inhibits the triggering of sarcomer contraction and modulate increase in cystolic pressure.

In one embodiment, calcium channel blockers, are amlodipine, aranidipine, barnidipine, benidipine, cilnidipine, clentiazem, diltiazen, efonidipine, fantofarone, felodipine, isradipine, lacidipine, lercanidipine, manidipine, mibefradil, nicardipine, nifedipine, nilvadipine, nisoldipine, nitrendipine, semotiadil, veraparmil, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry, which can be used in the compositions and methods of the invention.

In another embodiment, the β-blocker used in the compositions and methods of the invention is propanalol, terbutalol, labetalol propranolol, acebutolol, atenolol, nadolol, bisoprolol, metoprolol, pindolol, oxprenolol, betaxolol or a combination thereof.

In one embodiment, angiotensin II receptor blocker (ARB) are used in the compositions and methods of the invention. Angiotensin II receptor blocker (ARB) refers in one embodiment to a pharmaceutical agent that selectively blocks the binding of AII to the AT₁ receptor. ARBs provide in another embodiment, a more complete blockade of the RAAS by preventing the binding of AII to its primary biological receptor (AII type 1 receptor [AT₁]).

In another embodiment, the ARB used in the methods and compositions of the invention is candesartan, eprosartan, irbesartan losartan, olmesartan, telmisartan, valsartan or a combination thereof.

In one embodiment, a diuretic is used in the methods and compositions of the invention. In another embodiment, the diuretic is chlorothiazide, hydrochlorothiazide, methylclothiazide, chlorothalidon, or a combination thereof.

In one embodiment, the additional agent used in the compositions provided herein is a non-steroidal anti-inflammatory drug (NSAID). In another embodiment, the NSAID is sodium cromoglycate, nedocromil sodium, PDE4 inhibitors, leukotriene antagonists, iNOS inhibitors, tryptase and elastase inhibitors, beta-2 integrin antagonists and adenosine 2a agonists. In one embodiment, the NSAID is ibuprofen; flurbiprofen, salicylic acid, aspirin, methyl salicylate, diflunisal, salsalate, olsalazine, sulfasalazine, indomethacin, sulindac, etodolac, tolmetin, ketorolac, diclofenac, naproxen, fenoprofen, ketoprofen, oxaprozin, piroxicam, celecoxib, and rofecoxiband a pharmaceutically acceptable salt thereof. In one embodiment, the NSAID component inhibits the cyclo-oxygenase enzyme, which has two (2) isoforms, referred to as COX-1 and COX-2. Both types of NSAID components, that is both non-selective COX inhibitors and selective COX-2 inhibitors are useful in accordance with the present invention.

In another embodiment, the additional agent administered as part of the compositions, used in the methods provided herein, is a glycation inhibitor, such as pimagedine hydrochloride in one embodiment, or ALT-711, EXO-226, KGR-1380, aminoguanidine, ALT946, pyratoxanthine, N-phenacylthiazolium bromide (ALT766), pyrrolidinedithiocarbamate or their combination in yet another embodiment.

In one embodiment an antioxidant alone provides effective therapy. In another embodiment vitamin E alone provides effective therapy. In another embodiment a Gpx mimetic of any compound of Formulae (I)-(X) alone provides effective therapy.

As will be seen in the examples below, effective therapy for treating Hp 2 individuals is provided by combinations comprising an antioxidant. In another embodiment, the invention provides a composition comprising a statin; and a vitamin E or its derivative metabolite, or analog and/or their combination. In one embodiment, the invention provides a pharmaceutical composition comprising a statin; and a vitamin E or its derivative metabolite, or analog and/or their combination; and a diluent or carrier. In another embodiment, the statin is atorvastatin, rosuvastatin, fluvastatin, simvastatin, or cerivastatin. In another embodiment, the vitamin E or its derivative, metabolite, or analog and their combination is vitamin E. In another embodiment the composition or pharmaceutical composition comprises the combination of vitamin E and atorvastatin.

In another embodiment, the invention provides a composition comprising: a statin; and a glutathione peroxidase (GPx); its mimetic, isomer, functional derivative, synthetic analog, pharmaceutically acceptable salt or combination thereof. In one embodiment, the invention provides a pharmaceutical composition comprising: a GPx or its mimetic, isomer, functional derivative, synthetic analog, pharmaceutically acceptable salt or combination thereof; and a vitamin E or its derivative metabolite, or analog and their combination. In another embodiment, the statin is atorvastatin, rosuvastatin, fluvastatin, simvastatin, or cerivastatin. In another embodiment, the Gpx mimetic is any compound of Formula (I)-(X) herein. In another embodiment the Gpx mimetic is the compound of Formula (I). In another embodiment the composition or pharmaceutical composition comprises a combination of atorvastatin and the compound of Formula (I).

In another embodiment, the invention provides a composition comprising: a vitamin E or its derivative metabolite, or analog and/or their combination; and a glutathione peroxidase (GPx); its mimetic, isomer, functional derivative, synthetic analog, pharmaceutically acceptable salt or combination thereof. In one embodiment, the invention provides a pharmaceutical composition comprising: a GPx or its mimetic, isomer, functional derivative, synthetic analog, pharmaceutically acceptable salt or combination thereof; and a vitamin E or its derivative, metabolite, or analog and their combination. In one embodiment the vitamin E or its derivative, metabolite, or analog and their combination is vitamin E. In another embodiment, the Gpx mimetic is any compound of Formula (I)-(X) herein. In another embodiment the Gpx mimetic is the compound of Formula (I). In another embodiment the composition or pharmaceutical composition comprises a combination of vitamin E and the compound of formula I.

Accordingly and in another embodiment, herein is provided a method of determining the potential of a subject having cardiovascular disorder in a diabetic subject to benefit from administration of a composition comprising Vitamin E or its derivative metabolite, or analog and their combination; and a statin, or a composition comprising Vitamin E or its derivative metabolite, or analog and their combination; and a GPx or its isomer, functional derivative, synthetic analog, pharmaceutically acceptable salt or combination thereof in another embodiment; or a composition comprising a statin and a GPx or its isomer, functional derivative, synthetic analog, pharmaceutically acceptable salt or combination thereof, comprising the step of determining a haptoglobin phenotype of the subject, whereby a subject having a haptoglobin 2-2 phenotype will benefit from administration of any one of the compositions described herein

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

The term “preventing” refers in another embodiment, to preventing the onset of clinically evident pathologies associated with vascular complications altogether, or preventing the onset of a pre-clinically evident stage of pathologies associated with vascular complications in individuals at risk, which in one embodiment are subjects exhibiting the Hp-2 allele. In another embodiment, the determination of whether the subject carries the Hp-2 allele, or in one embodiment, which Hp allele, precedes the methods and administration of the compositions of the invention.

Cardiovascular disease (CVD) is the most frequent, severe and costly complication of type 2 diabetes. It is the leading cause of death among patients with type 2 diabetes regardless of diabetes duration. In one embodiment, allelic polymorphism contributes to the phenotypic expression of CVD in diabetic subjects. In another embodiment, the methods and compositions of the invention are used in the treatment of CVD in diabetic subjects.

The term “myocardial infarct” or “MI” refers in another embodiment, to any amount of myocardial necrosis caused by ischemia. In one embodiment, an individual who was formerly diagnosed as having severe, stable or unstable angina pectoris can be diagnosed as having had a small MI. In another embodiment, the term “myocardial infarct” refers to the death of a certain segment of the heart muscle (myocardium), which in one embodiment, is the result of a focal complete blockage in one of the main coronary arteries or a branch thereof. In one embodiment, subjects which were formerly diagnosed as having severe, stable or unstable angina pectoris, are treated according to the methods or in another embodiment with the compositions of the invention, upon determining these subjects carry the Hp-2 allele and are diabetic.

The term “ischemia-reperfusion injury” refers in one embodiment to a list of events including: reperfusion arrhythmias, microvascular damage, reversible myocardial mechanical dysfunction, and cell death (due to apoptosis or necrosis). These events may occur in another embodiment, together or separately. Oxidative stress, intracellular calcium overload, neutrophil activation, and excessive intracellular osmotic load explain in one embodiment, the pathogenesis and the functional consequences of the inflammatory injury in the ischemic-reperfused myocardium. In another embodiment, a close relationship exists between reactive oxygen species and the mucosal inflammatory process.

In another embodiment, the route of administration in the step of contacting in the methods of the invention, using the compositions described herein, is optimized for particular treatments regimens. If chronic treatment of cardiovascular complications is required, in one embodiment, administration will be via continuous subcutaneous infusion, using in another embodiment, an external infusion pump. In another embodiment, if acute treatment of vascular complications is required, such as in one embodiment, in the case of miocardial infarct, then intravenous infusion is used.

In one embodiment, the compositions provided herein are administered in conjunction with other therapeutical agents. Representative agents that can be used in combination with the compositions of the invention are agents used to treat diabetes such as insulin and insulin analogs (e.g. LysPro insulin); GLP-1 (7-37) (insulinotropin) and GLP-1 (7-36)-NH.sub.2; biguanides: metformin, phenformin, buformin; .alpha.2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glypizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; .alpha.-glucosidase inhibitors: acarbose, miglitol, emiglitate, voglibose, MDL-25,637, camiglibose, MDL-73,945; .beta.-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan.RTM.)) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (Symlin.TM.), AC2993, glycogen phosphorylase inhibitor and nateglinide. Any combination of agents can be administered as described hereinabove.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Methods

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Haptoglobin Phenotyping: Haptoglobin phenotyping was determined from 10 .mu.l of EDTA-plasma by gel electrophoresis and peroxidase staining using a modification.sup.44,45 of the method originally described by Smithies.sup.46 which used starch gel electrophoresis and peroxidase staining with benzidine. Patients' plasma was stored at −20.degree. C. All chemicals were purchased from Sigma Israel (Rehovot, Israel). A 10% hemoglobin solution in water was prepared from heparinized blood by first washing the blood cells 5 times in phosphate buffered saline and then lysing the cells in 9 ml of sterile water per ml of pelleted cell volume. The cell lysate was centrifuged at 10,000 g for 40 minutes and the supernatant containing hemoglobin was aliquoted and stored at −70.degree. C. Serum (10 .mu.l) was mixed with 2 .mu.l of the 10% hemoglobin solution and the samples permitted to stand for 5 minutes at room temperature in order to allow the haptoglobin-hemoglobin complex to form. An equal volume (12 .mu.l) of sample buffer containing 125 mM Tris Base pH 6.8, 20% (w/v) glycerol and 0.001% (w/v) bromophenol blue was added to each sample prior to running on the gel. The haptoglobin hemoglobin complex was resolved by polyacrylamide gel electrophoresis using a buffer containing 25 mM Tris Base and 192 mM glycine. The stacking gel was 4% polyacrylamide (29:1 acrylamide/bis-acrylamide) in 125 mM Tris Base, pH 6.8 and the separating gel was 4.7% polyacrylamide (29:1 acrylamide bis-acrylamide) in 360 mM Tris Base, pH 8.8. Electrophoresis was performed at a constant voltage of 250 volts for 3 hours. After the electrophoresis was completed the haptoglobin-hemoglobin complexes were visualized by soaking the gel in freshly prepared staining solution in a glass tray. The staining solution (prepared by adding the reagents in the order listed) contained 5 ml of 0.2% (w/v) 3,3′,5,5′-tetramethylbenzidine in methanol, 0.5 ml dimethylsulfoxide, 10 ml of 5% (v/v) glacial acetic acid, 1 ml of 1% (w/v) potassium ferricyanide and 150 .mu.l of 30% (w/w) hydrogen peroxide. The bands corresponding to the haptoglobin-hemoglobin complex were readily visible within 15 minutes and were stable for over 48 hours. All gels were documented with photographs. The haptoglobin phenotype of all samples was determined at the laboratory without any knowledge concerning the patient.

Plasma samples were received by the laboratory for analysis and haptoglobin phenotyping was possible on all but six of these samples. For these six patients it is not clear if they represent patients who do not make any haptoglobin (Hp 0 phenotype).sup.22,23 or that the haptoglobin concentration is below the detection limit for the assay described.

Human Blood Products. All protocols in this study were approved by the Institutional Review Boards of participating centers. All individuals provided informed consent. Serums used in this study were obtained from outpatient clinics at the Rambam Medical Center and the Haifa and Western Galilee district of Clalit Health Services.

Chemicals and Reagents. All reagents were from Sigma Israel (Rehovot) unless otherwise indicated. Radiochemicals were purchased from Amersham. Materials for cell culture were purchased from Biological Industries (Bet Haemek). Hp was purified from healthy volunteers by antibody affinity chromatography. The Hp concentration of purified Hp was determined spectrophotometrically using the known extinction coefficients of Hp (53.9 for Hp1-1 and 58.65 for Hp2-2). The Hp molar concentration was calculated based on the monomer of each Hp type. HDL was prepared from the serum of fasted normolipidemic normal human volunteers by density gradient ultracentrifugation as previously described.

Biochemical Measurements. Serum cholesterol was assayed using commercially available enzymatic-colorimetric methods (Roche Chol CHOD-PAP). HDL in human serum was assayed after sodium phophotungstate-Mg2 precipitation. HDL in murine serum was assayed by ELISA (Bio-Systems). Hp in human serum was measured immunonephelometrically and in murine serum by ELISA (Mercodia).

Isolation and Glycation of Hb. Native Hb was isolated from fresh human blood. Hb concentrations were calculated using the Bradford reagent. Hb was glycated in vitro using glycolaldehyde.

Measurement of HDL-Associated Lipid Peroxides. Glycosylated or nonglycosylated Hb (1 umol/L) was incubated with 100 ug of HDL and 20 umol/L ascorbic acid in PBS with or without Hp1-1 or Hp2-2 (equimolar to Hb) for 3 hours at 37° C. Lipid peroxides were measured as previously described.

Determination of the Hp Genotype. The Hp genotype of participants in this study was determined by nondenaturing gel electrophoresis and peroxidase staining, using a modification of a previously described method.

Cell Culture. J774 A.1 murine macrophage cells were purchased from the American Type Culture Collection (Manassas, Va.) and grown in DMEM supplemented with 5% FBS.

Cholesterol Efflux From Macrophages. Murine J774 cells (1×10⁶/mL) were plated in 24-well plates for 48 hours, then washed and incubated in DMEM without serum containing ³H-cholesterol (2 uCi/mL) for 1 hour. Cells were washed to remove unincorporated label and then incubated in 1 mL of DMEM supplemented with: (1) nothing (negative control); (2) purified HDL (100 ug/mL protein) (positive control); or (3) 30 uL of serum from individuals with or without DM with the different Hp genotypes. In studies using purified Hp and Hb rather than serum, the cells were incubated with purified HDL (50 uL/mL protein) with different combinations of native Hb, glycated Hb, Hp1-1, and Hp2-2 (all at 0.8 umol/L).

After a 3-hour incubation at 37° C. to permit efflux of ³H-cholesterol from the cells into the medium, 500 uL of the medium was collected, the cells washed with PBS, and 0.1 N NaOH added to the cells. Cellular and medium ³H-cholesterol were determined by liquid scintillation counting (LSC). The percentage of cholesterol efflux was calculated as the ratio of total counts per minute in the medium divided by the total counts per minute in the medium and in the cells. HDL-mediated cholesterol efflux (resulting from purified HDL or HDL found in the serum) was calculated after subtraction of the nonspecific efflux obtained in cells incubated in the absence of purified HDL or serum. Results reported for efflux elicited by serum samples are normalized for the serum HDL concentration derived as (measured efflux)(measured HDL in mg/dL)/50.

Determination of LCAT Cholesterol Esterification Rate in Serum. LCAT cholesterol esterification rate in serum was measured using the method of Ohta et al. Briefly, 0.25 uCi of ³H-free cholesterol (³H-FC) was added to a 1:5 dilution of serum (500 uL of total volume) and incubated at 37° C. for 90 minutes. The enzyme reaction catalyzing the esterification of FC was stopped by immersing the sample tubes in an ice bath. Lipids were extracted with n-hexane:isopropanol 3:2 (vol/vol), dried under nitrogen and resuspended in chloroform. Lipid extract was spotted on thin-layer chromatography plates and developed in n-hexane:diethyl ether:acetic acid:methanol (85:20:1:1) (vol/vol). Spots corresponding to FC and cholesterol ester were cut out from the plates and the radioactivity was determined by LSC. The fractional esterification rate (FER) was expressed as the difference between the percentage of radioactive cholesterol esterified before and after incubation at 37° C. and the molar esterification rate was calculated based on the specific activity (counts per minute per nanomole of FC) of each sample. Results reported for FER in the serum samples are normalized for the serum HDL concentration derived as (measured FER)(measured HDL in mg/dL)/50.

In Vivo Studies. Mice. Mice were housed and procedures approved according to the guidelines of the Animal Care and Use Committee of the Technion. All mice used in this study had a C57B1/6 genetic background. The Hp2 allele exists only in humans. The C57B1/6 wild-type murine Hp gene is a class 1 allele with more than 90% homology to the human class 1 Hp allele. A murine Hp2 allele was created by molecular engineering of the murine Hp1 allele as described in the online data supplement. The murine Hp2 allele was targeted for insertion at the murine Hp locus by homologous recombination resulting in a replacement of the wild-type Hp1 allele with a murine Hp2 allele. The generation of Hp2-2 mice after this targeted insertion is described in the online data supplement. Characterization of haptoglobin in Hp2-2 mice by gel electrophoresis demonstrated that the distribution of Hp polymers in Hp2-2 mice was similar to that in Hp2-2 humans.

Diabetes mellitus (DM) was induced by intraperitoneal injection of streptozotocin (200 mg/kg) dissolved in 50 mmol/L citrate buffer (pH 4.5) at 6 weeks of age. Glucose levels were monitored with a glucometer and HbAlc was measured using a diagnostic kit from Sigma. Mice were 1420 Circulation Research Dec. 8/22, 2006 fed a standard chow diet (Teklad-Harlan, Certified Global 18% Protein Rodent Diet; catalog no. 2018SC+F). DM and non-DM littermates followed in parallel were used for these studies.

Measurement of RCT. We used a recently described method for measuring RCT in mice. Male C57BL/6 mice at the age of 9 weeks (DM duration of 3 weeks) were used for this study. Each animal was caged separately with unlimited access to food and water. J774 cells were cultured in DMEM supplemented with 5% FBS, 5 uCi/mL ³H-cholesterol, and 30 ug/mL acetylated LDL for 48 hours. Cells were washed twice and cellular associated radioactivity determined. The ratio of radiolabeled FC and radiolabeled cholesterol ester in these cells was assessed by thin-layer chromatography, and more than 70% of the ³H-cholesterol incorporated into J774 foam cells was esterified. ³H-Cholesterol-labeled and cholesterol-loaded J774 foam cells were injected intraperitoneally into Hp 1-1 or Hp2-2 mice with or without DM (4×10⁶ cells containing 4.5×10⁶ cpm in 0.5 mL medium for each mouse). Mice were bled at 24 hours (from the retroorbital plexus) and at 48 hours (from the inferior vena cava). Blood was used for LSC and for lipid analysis. At 48 hours, mice were euthanized and liver tissue stored at −20° C. until lipid extraction was performed. Feces were collected continuously more than the study interval and were stored at 4° C. until cholesterol and bile acid extraction were performed.

Tissue Lipid Extraction. Tissue lipids from 100 mg of homogenized liver tissue were extracted twice with n-hexane and isopropanol 3:2 (vol/vol), evaporated under nitrogen, dissolved in chloroform, and counted by LSC. The distribution of radioactive FC and cholesterol ester in liver tissue was assessed by thin-layer chromatography.

Fecal Cholesterol and Bile Acid Extraction. Fecal cholesterol and bile acids were extracted from the feces as previously described. Briefly, the total feces collected over the 48-hour study period were soaked in water for 16 hours (1 mL per 100 mg of feces). An equal volume of ethanol was then added and the mixture homogenized. Total ³H-sterols was determined by taking 400 uL of the homogenized feces and counting in LSC. To extract the ³H-cholesterol from homogenized feces, 2 mL of the homogenized feces was mixed with an equal volume of ethanol followed by the addition of 500 uL of 1 mol/L NaOH and the samples saponified at 95° C. for 2 hours. This homogenate was then extracted 3 times with hexane, evaporated under nitrogen, and resuspended with chloroform, and the ³H-cholesterol was counted in LSC. To measure ³H-bile acids, the feces solution was acidified with concentrated HCl, extracted 3 times with ethyl acetate, evaporated under nitrogen, resuspended in ethyl acetate and counted by LSC.

Hp transgenic mice and veneration of DM. All protocols were approved by the Animal Care and Use Committee of the Technion. Sprague Dawley rats (6 wk) were purchased from Harlan Labs. The characterization and generation of Hp 1 and Hp 2 mice in a C57B1/6 genetic background has previously been described in detail. Briefly, the murine Hp 1 allele is the wild type murine Hp allele. The Hp 2 allele exists only in man and a murine Hp 2 allele was genetically engineered in vitro and its insertion targeted at the Hp locus by homologous recombination. Characterization of these mice has demonstrated that there are no differences in plasma biochemical and lipid parameters. Diabetes was generated in these mice at 8 weeks of age using a low dose streptozotocin protocol (NIH Funded Consortium on Animal Models of Diabetic Complications protocol available on the consortium web site www.amdcc.org) and confirmed by measuring plasma glucose. Mice were diabetic for at least 1 month prior to analysis.

Analysis of the half-life of the Hp-Hb complex in vivo Human Hp 1-1 and Hp 2-2 were affinity purified and human Hb was prepared from lysed red blood cells. Hp was labeled with ¹²⁵I by the chloramine T method as previously described. Labeled Hp 1-1 or Hp 2-2 was complexed with a two fold molar excess of Hb and 40 ng of the complex was injected in the tail vein of either Hp 1 or Hp 2 mice with or without DM in a total volume of 200 microliters with a 23 g needle. The half-life of the Hp 1 and Hp 2-Hb complexes were measured in both Hp 1 and Hp 2 mice. Blood was taken from the retroorbital plexus at multiple time points (1 minutes-180 minutes) after the injection of the label and cpm determined in a gamma counter.

Immunoprecipitation of HDL in mice injected with ¹²⁵I labeled Hp-Hb Serum from mice previously injected with ¹²⁵I labeled Hp-Hb was used for these studies. Serum was first cleared with protein A/G sepharose (DAKO) and then incubated with a rabbit polyclonal serum (200 ug/ml) to mouse apolipoprotein A1 (DAKO) at a 1:20 dilution. Immunoprecipitation was then achieved with protein A/G sepharose. The pellet was washed three times with washing buffer (150 mM NaCl, 50 mM Tris pH 8.0, 1% Triton). The amount of cpm in the immunoprecipitate was determined in a gamma counter.

Statistical analysis All results are reported as the mean±SME. Pairwise comparison between groups was performed using Student's t test, with a probability value of <0.05 considered statistically significant.

### Example 1

### Impaired Cholesterol Efflux from Macrophages Elicited by Serum from Hp2 DM Individuals

We sought to determine whether there were differences in cholesterol efflux from macrophages incubated with serum from 90 DM and 72 non-DM individuals segregated by Hp genotype. Patients included in this analysis were randomly selected from a larger cohort of individuals from whom stored sera were available to ensure an equal distribution of the three Hp genotypes. Consistent with previous reports, we found that the serum Hp concentration was Hp-type dependent, with significantly mean higher values in Hp1-1 and lower mean values in Hp2-2. The serum Hp concentration segregated by Hp genotype in the DM cohort was 1.78±0.34 mg/mL for Hp1-1 individuals, 1.92±0.11 mg/mL for Hp2-1 individuals, and 1.25±0.08 mg/mL for Hp2-2 individuals. In the non-DM cohort, the Hp concentration was 1.75±0.12 mg/mL for Hp1-1 individuals, 1.47±0.09 mg/mL for Hp2-1 individuals, and 1.16±0.12 mg/mL for Hp2-2 individuals. There were no significant differences between the Hp types in demographic characteristics (i.e., age, gender), comorbid conditions, or lipid parameters (total cholesterol, HDL).

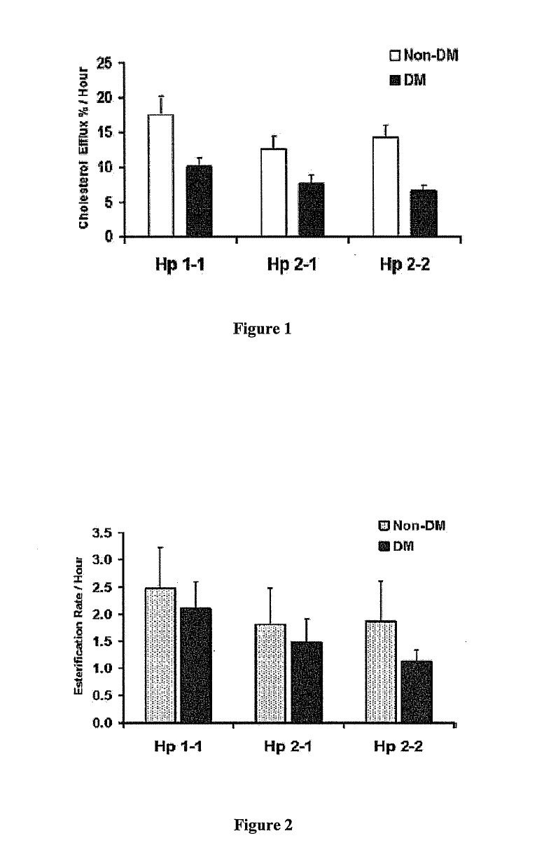

We found that there were no significant differences in cholesterol efflux from J774 cells incubated with serum from non-DM individuals with the Hp1-1 (n=22), Hp2-1 (n=26), or Hp2-2 (n=24) genotypes. Incubation of J774 cells with serum from DM individuals resulted in a significant reduction in the cholesterol efflux compared with cells incubated with serum from non-DM individuals (14.84±1.85% versus 8.1±1.12% for non-DM versus DM individuals; P<0.001). The reduction in cholesterol efflux associated with DM serum was Hp-type dependent. Efflux elicited with serum from DM Hp1-1 (n=30) individuals was significantly higher as compared with efflux elicited with serum from DM Hp2-1 (n=30) or Hp2-2 (n=30) individuals (P<0.01) (FIG. 1).

### Example 2

### LCAT Cholesterol Esterification Rate is Markedly Reduced in Diabetic Patients with the Hp2 Allele

We sought to determine whether there were any differences in the LCAT cholesterol esterification rate in the diabetic state and whether LCAT cholesterol esterification rate was associated with the Hp type. We measured LCAT cholesterol esterification rate in the serum of 84 DM and 62 non-DM individuals with Hp1-1, Hp2-1, and Hp2-2 (the same patients in whom cholesterol efflux was measured: Example 1 and FIG. 1). We found a pattern similar to what was observed for cholesterol efflux. In non-DM individuals there were no differences in LCAT cholesterol esterification rate according to the Hp type (FIG. 2). In DM individuals, we found that the highest LCAT cholesterol esterification rate was observed in Hp1-1 individuals, the lowest in Hp2-2 individuals, and an intermediate level in Hp2-1 individuals. In the Hp1-1 group, there was no significant difference in LCAT cholesterol esterification rate between DM and non-DM individuals.

### Example 3

### Decreased Cholesterol Efflux from Macrophages Incubated with Glycated Hb and Hp2-2

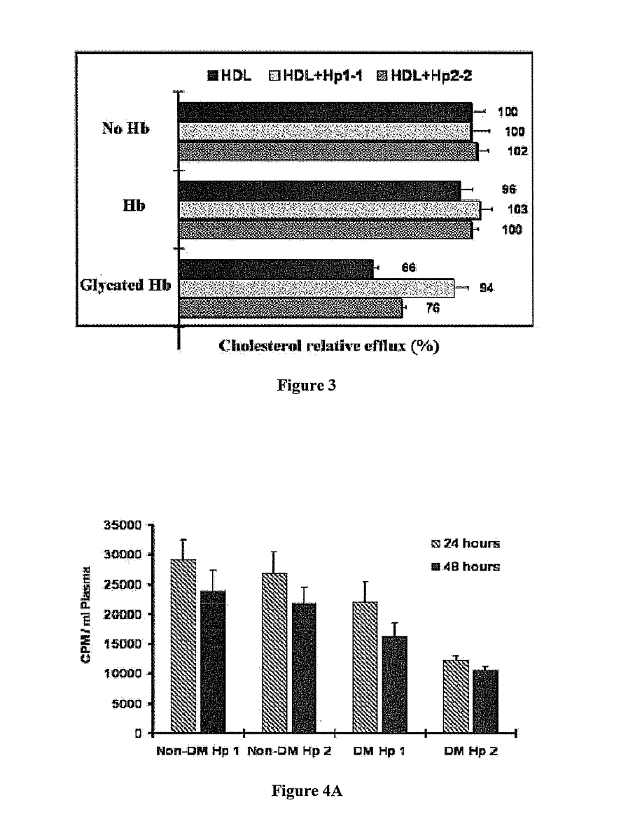

We sought to examine whether the reduction in the cholesterol efflux from cells elicited by serum from DM individuals with the Hp2 allele could be recapitulated using purified Hp and Hb. We found that the addition of Hp1-1, Hp2-2, or native Hb did not cause any reduction in the HDL-mediated efflux of ³H-cholesterol. However, the addition of glycated Hb resulted in a significant 35% reduction in HDL-mediated cholesterol efflux (P<0.001). Hp1-1 was able to block the glycated Hb impairment in HDL-mediated cholesterol efflux by more than 80±6% as compared with only 30±4% with Hp2-2 (P<0.001) (FIG. 3).

These foregoing observations can be explained by differences in the oxidation of proteins or lipids involved in cholesterol efflux. To demonstrate that glycosylated Hb can oxidatively modify molecules involved in the efflux process within the time frame of this experiment (3 hours), we assessed the ability of glycosylated and nonglycosylated Hb to oxidize HDL associated lipids. We found a marked increase (mean of 142.3 nmol of lipid peroxide per milligram of HDL in 2 independent experiments) in lipid peroxides when HDL was incubated with glycated Hb for 3 hours, whereas no increase in HDL associated lipid peroxides was found over this interval when using nonglycated Hb. Furthermore, Hp1-1 nearly completely blocked the ability of glycated Hb to induce HDL-associated lipid peroxides (mean inhibition of 94% in 2 independent experiments), whereas Hp2-2 had only a partial inhibitory activity (50% in 2 independent experiments).

### Example 4

### RCT is Dramatically Decreased In Vivo in Diabetic Mice in a Hp-Dependent Manner

We injected 3H-cholesterol-labeled J774 macrophages into the peritoneum of 16 DM and non-DM Hp1-1 or Hp2-2 mice (n=4 for each subgroup). The lipid profile and diabetes characteristics of these mice are provided in Table 1 below.

TABLE 1
Lipid Profile and DM Characteristics of Mice

| Hp Type | n | DM | Age (wk) | Hp (mg/mL) | Glu (mg/dL) | HbA1c | Total Cholesterol (mg/dL) | HDL (mg/dL) |
|---|---|---|---|---|---|---|---|---|
| Hp1-1 | 4 | + | 9 | 1.4 ± 0.5 | 482 ± 53 | 11.2 ± 1.1 | 214 ± 48 | 56.5 ± 5.5 |
| Hp2-2 | 4 | + | 9 | 1.4 ± 0.6 | 487 ± 94 | 12.3 ± 1.3 | 194 ± 36 | 49.6 ± 3.1 |
| Hp1-1 | 4 | − | 9 | 1.6 ± 0.5 | 130 ± 19 | | 196 ± 39 | 60.2 ± 3.5 |
| Hp2-2 | 4 | − | 9 | 1.2 ± 0.4 | 123 ± 9 | | 191 ± 22 | 62.6 ± 5.8 |

Hp, glucose (Glu), HbA1c, total cholesterol, and HDL are presented as mean ± SEM. No significant differences in total cholesterol or in HDL levels were found among the different groups.

There was no significant difference in either the total or HDL cholesterol among any of the 4 subgroups. Glucose and HbA1c were not significantly different between DM mice with the Hp1-1 and Hp2-2 genotypes (Table). Furthermore, we found no difference in the serum Hp concentration between Hp1-1 and Hp2-2 mice in the presence or absence of DM (Table 1).

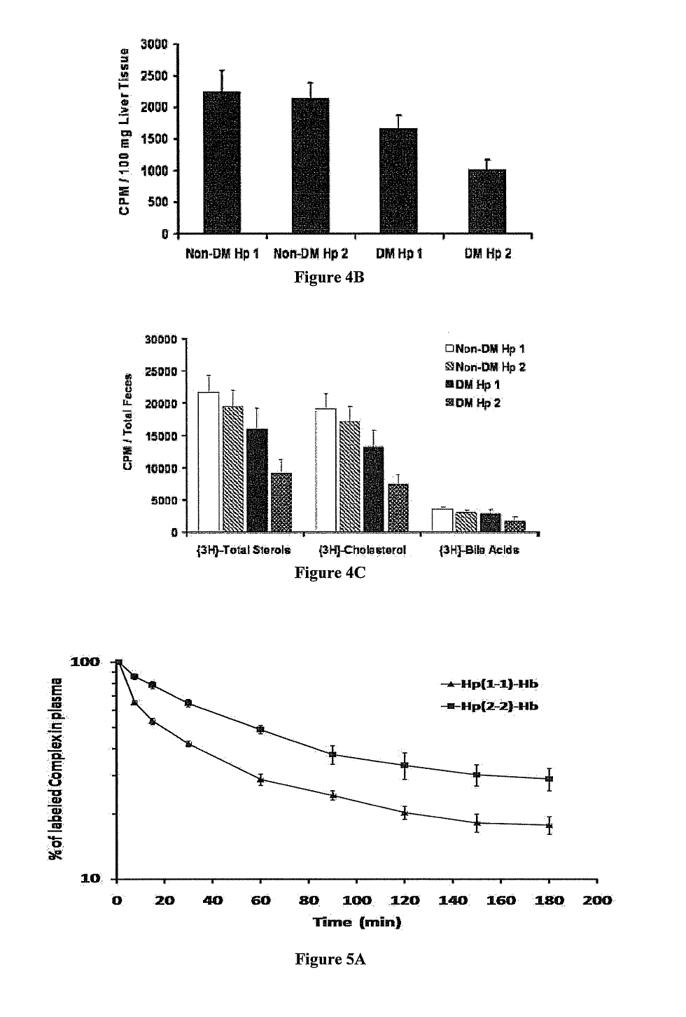

There were no significant differences in plasma, liver, or fecal ³H-cholesterol between the non-DM mice with the different Hp types (P=0.2). In DM mice as compared with non-DM mice, we found a 38±10% reduction in the appearance of 3H-cholesterol in plasma as compared with non-DM mice at 24 hours and a 41±11% reduction at 48 hours after injection of the J774 cells (P<0.012) (FIG. 4A). We found striking Hp-type differences in the amount of ³H-cholesterol in the plasma, liver, and feces in DM mice (FIG. 4). The reduction in ³H-cholesterol associated with DM was significantly greater in Hp2-2 mice as compared with Hp1-1 mice (54±9% versus 25±13% in plasma [FIG. 4A]; 52±10% versus 27±14% in liver [FIG. 4B]; 57±10% versus 32±10% in feces [FIG. 4C]; P<0.03). ³H-bile acids levels were not significantly different among the groups.

### Example 5

### The Half-Life of the Hp 2-Hb Complex is Greater than the Hp 1-Hb Complex In Vivo

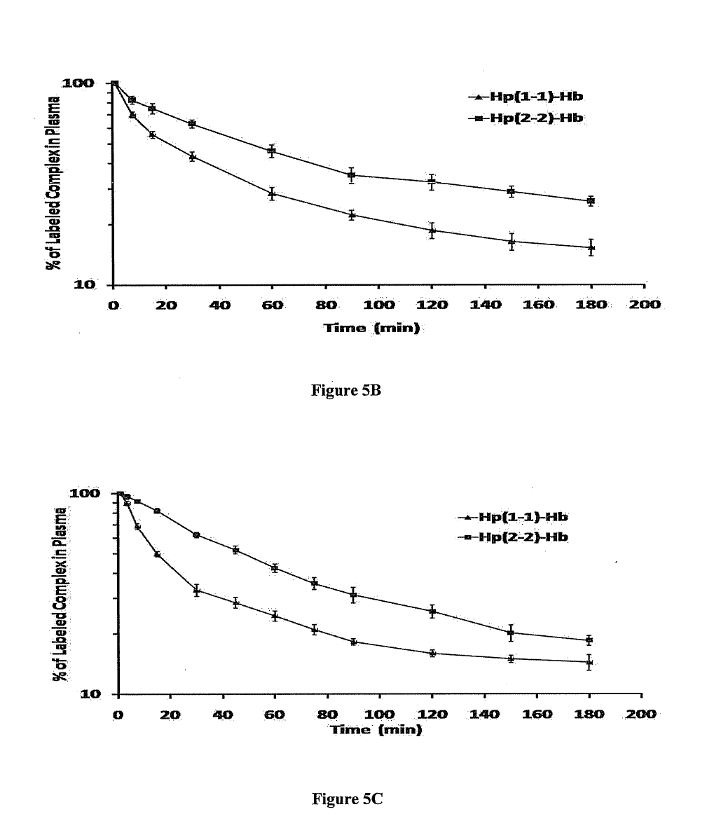

The half-life of Hp 1-Hb and Hp 2-Hb complex were measured in Hp 1 and Hp 2 mice and in Sprague-Dawley rats (Hp 1) (FIGS. 5A, B and C, respectively). The decay of the injected counts followed a biphasic course. The initial rapid phase of decay was significantly longer with the Hp 2 as compared to the Hp 1-Hb complex. Similar results for this rapid phase of clearance were seen in Hp 1 mice, Hp 2 mice and in Sprague-Dawley rats (Table 2). The rapid phase of clearance was followed by a slower phase of clearance which was not significantly different between the Hp 1-Hb and Hp 2-Hb complexes.

TABLE 2
Half-life of the Hp 1 and Hp 2 complex in non-DM mice and rats.

| Animal strain | N | Hp-Hb complex | Half-Life (min) |
|---|---|---|---|
| Hp 1 mice | 5 | Hp 1 | 20.4 ± 3.8 |
| Hp 1 mice | 5 | Hp 2 | 57.8 ± 6.2 |
| Hp 2 mice | 5 | Hp 1 | 24.5 ± 4.0 |
| Hp 2 mice | 5 | Hp 2 | 53.8 ± 7.3 |
| Rat | 4 | Hp 1 | 17.3 ± 4.1 |
| Rat | 4 | Hp 2 | 48.0 ± 7.5 |

### Example 6

### The Half-Life of the Hp 2-Hb Complex is Markedly Increased in DM

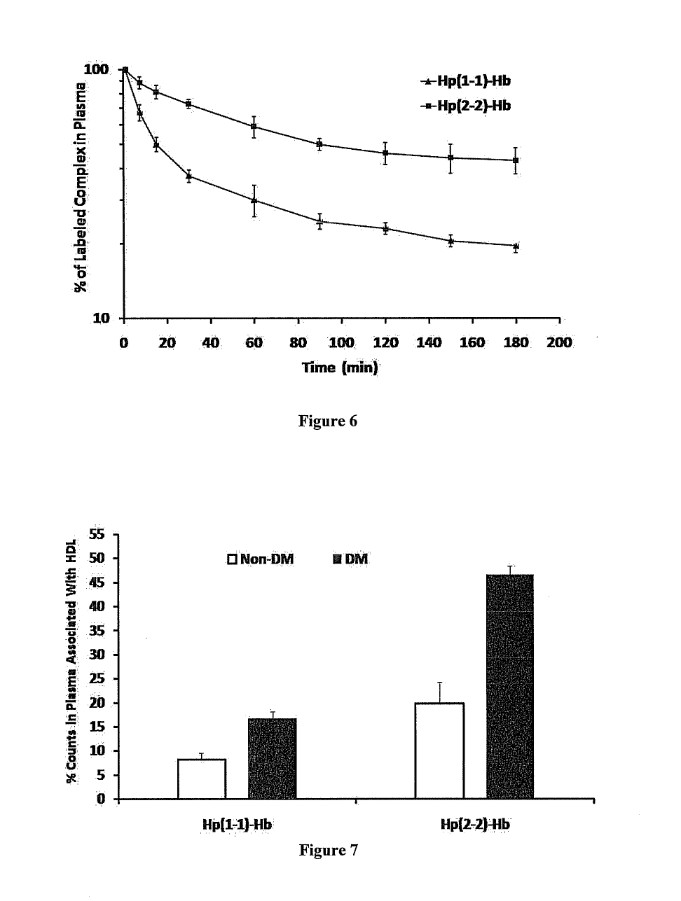

We measured the half-life of the Hp 1-Hb and Hp 2-Hb complex in DM Hp 1 and DM Hp 2 mice. We found that DM resulted in a marked decrease in the initial rate of clearance of the Hp 2-Hb complex but had no effect on the initial rate of clearance of the Hp 1-Hb complex (FIG. 6 and Table 3).

TABLE 3
Half-life of the Hp 1 and Hp 2 complex in DM mice

| Animal strain | N | Hp-Hb complex | Half-Life (min) |
|---|---|---|---|
| Hp 1 mice | | Hp 1 | |
| Hp 1 mice | | Hp 2 | |
| Hp 2 mice | 6 | Hp 1 | 18.6 ± 4.5 |
| Hp 2 mice | 6 | Hp 2 | 103 ± 9.5 |

### Example 7

### Increased Association of Hp 2-Hb with HDL in DM Mice

The amount of ¹²⁵I-Hp-Hb associated with HDL was measured by immunoprecipitating HDL in mice that had been injected with ¹²⁵I-Hp-Hb. Whereas the total number of cpm in plasma declined in a biphasic manner as demonstrated in FIG. 5 in Example 6, the total number of cpm associated with HDL as detected by coimmunoprecipitation did not change significantly over the interval that the decay of the complex was monitored. The percentage of the injected cpm coimmunoprecipitated with HDL at all time points measured was 2.1±0.3% for the Hp 1-1-Hp complex in non DM mice (n=5), 8.1±1.7% for the Hp 2-2-Hp complex in non-DM mice (n=5), 4.5±0.4% for the Hp 1-1-Hb complex in DM mice (n=6), and 25.6±1.2% for the Hp 2-2-Hb complex in DM mice (n=6). Because the percentage of the injected labeled Hp-Hb complex associated with HDL remained stable while the total amount of labeled Hp-Hb in plasma declined rapidly, the fraction of plasma Hp-Hb which was found to be associated with HDL increased with time after injection. As demonstrated in FIG. 7 in Hp 2 mice, 75 minutes after injection of the complex, a significantly higher percentage of the total plasma Hp 2-Hb complex, as compared to the total plasma Hp 1-Hb complex, consisted of complex bound to HDL (19.8±4.4% vs. 8.3±1.3%, p=0.028). In Hp 2 DM mice, an even higher percentage of the total plasma Hp 2-Hb complex was found to be bound to HDL as compared to non DM Hp 2 mice (46.5±1.9%, p<0.0001 compared to Hp 2-Hb in non-DM mice). Similar results were obtained in Hp 1 mice.

### Example 8

### Impaired Cholesterol Efflux from Macrophages Elicited by Serum from Hp 2 DM Mice and Prevention of this Impairment by Vitamin E

A determination was sought, of whether there were differences in cholesterol efflux from macrophages incubated with serum from Hp 1 and Hp 2 mice with and without DM. There was no significant difference in the Hp concentration or in the lipid profile between mice with the different Hp genotypes with or without DM. There was no significant difference in glucose control between the Hp 1 and Hp 2 mice.

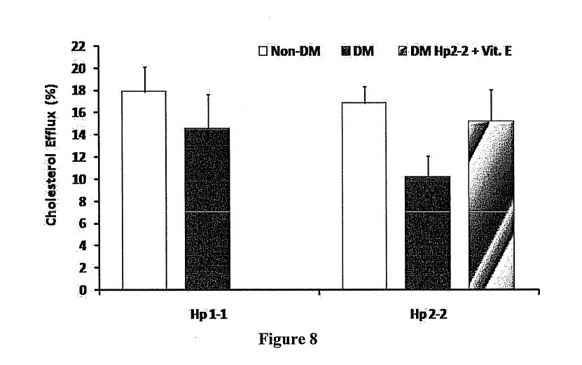

It was found that there were no significant differences in cholesterol efflux from J774 cells incubated with serum from non-DM mice with the Hp 1-1 or Hp 2-2 genotype (17.8±1.2% for Hp 1-1 mice vs. 16.8±0.8% for Hp 2-2 mice, n=10 for each group, p=0.50) (FIG. 8). Furthermore, no observation was made concerning any difference in cholesterol efflux due to DM in serum from Hp 1 mice (14.5±1.7% for Hp 1 DM mice vs. 17.8±1.2% for Hp 1 non DM mice, n=10 for each group, p=0.14). However, a highly significant reduction in cholesterol efflux due to DM was observed in serum from Hp 2 mice (10.2±1.1% for Hp 2 DM mice vs. 16.8±0.8% for Hp 2 non DM mice, n=10 for each group, p=0.0001) (FIG. 7).

Vitamin E (racemic, alpha-tocopherol acetate, Merck) was administered to a subset of the Hp 2-2 DM mice at a dose of 40 mg/kg/day beginning three weeks after the induction of DM. Vitamin E had no effect on glucose control or on the lipid profile. The cholesterol efflux from macrophages elicited by serum from Hp 2-2 DM was compared in mice that had or had not been treated with vitamin E. Vitamin E administration was found to prevent the impairment in efflux produced by DM in Hp 2 mice, as there was no significant difference in efflux elicited by serum from Hp 2 mice without DM and from Hp 2 DM mice treated with vitamin E (p=0.43) (FIG. 8).

### Example 9

### Dual Therapy with Statins and Antioxidants is Superior to Statins Alone in Decreasing the Risk of Cardiovascular Disease in Individuals with Diabetes Mellitus and the Haptoglobin 2-2 Genotype

The study protocol of the ICARE study involves the following: participants were drawn from 47 primary health clinics of the Clalit Health Services in the northern sector of Israel. Patients were eligible for the study if they had Type II DM and were 55 years of age or older. 3054 individuals underwent Hp genotyping and of these 1434 were found to have the Hp 2-2 genotype. These Hp 2-2 individuals were randomly assigned to treatment with either vitamin E or placebo. The major study outcomes (MI, stroke, CVD death) were identified prospectively in this population over an 18 month period. A preplanned secondary analysis of ICARE was to assess the ability of vitamin E therapy to influence outcomes in those ICARE participants who were also taking statins. Statin use as prospectively defined in ICARE was based on the use of statins by the participant in at least eight of the twelve months preceding enrollment of the participant in the study. The decision to use statins for a particular participant was under the discretion of the patient's primary care physician and was in no way influenced by the patient's participation in the ICARE study.

Robust clinical data has shown that individuals homozygous for the Hp 2 allele (Hp 2-2 genotype), 40% of DM individuals, have an up to 500% increased risk of CVD (21-24). A vast amount of basic science, animal and epidemiological data has provided the logic for targeting vitamin E administration specifically to DM individuals with the Hp 2-2 genotype. In the ICARE study (Israel CArdiovascular events Reduction with vitamin E (ClinicalTrials.gov# NCT00220831)) a prospective randomized placebo controlled trial of vitamin E therapy in DM individuals with the Hp 2-2 genotype, is was shown that vitamin E therapy results in a 50% reduction in CVD events. However, only about half of the Hp 2-2 DM participants in ICARE received statin therapy. Because statin therapy is currently recommended for all DM individuals we sought to determine if antioxidant therapy could still be demonstrated to provide benefit to Hp 2-2 DM individuals also taking statins in ICARE.

Of the 801 Hp 2-2 individuals taking statins in the ICARE cohort, 386 were randomized to vitamin E and 415 to placebo. There was no significant difference in the baseline characteristics, concurrent medications, or diabetes characteristics between those individuals taking statins who were randomized to placebo or vitamin E. It was found that dual treatment with statins and vitamin E dramatically reduced the event rate compared to statin treatment alone. (1.3% (5/386) for vitamin E vs. 4.1% (17/415) for placebo, hazard ratio [HR] 0.31, 95% confidence interval [CI] 0.15-0.83, p=0.017 by log-rank).

This magnitude of the beneficial affect of vitamins in those taking statins was unchanged if the definition for statin use was widened to include those patients taking statins in at least one of the twelve months preceding enrollment of the participant in the study (1.7% (9/538) for vitamin E vs. 4.2% (23/543) for placebo, p=0.013)

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of determining prognosis for a subject having a vascular complication, to benefit from treatment with reverse cholesterol transport therapy comprising the step of obtaining a biological sample from the subject; and determining the subject's haptoglobin allelic genotype, whereby a subject expressing the Hp-2-2 genotype will benefit from treatment with reverse cholesterol transport therapy.

2. The method of claim 1, wherein said step of determining said haptoglobin genotype is effected by a method selected from a signal amplification method, a direct detection method, detection of at least one sequence change, immunological method or a combination thereof.

3. The method of claim 2, wherein said signal amplification method amplifies a molecule selected from the group consisting of a DNA molecule and an RNA molecule.

4. The method of claim 2, wherein said signal amplification method is selected from the group consisting of PCR, LCR (LAR), Self-Sustained Synthetic Reaction (3SR/NASBA) and Q-Beta (Qβ) Replicase reaction.

5. The method of claim 2, wherein said direct detection method is selected from the group consisting of a cycling probe reaction (CPR) and a branched DNA analysis.

6. The method of claim 2, wherein said detection of at least one sequence change employs a method selected from the group consisting of restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis and Dideoxy fingerprinting (ddF).

7. The method of claim 2, wherein step of determining said haptoglobin genotype is effected by an immunological detection method.

8. The method of claim 7, wherein said immunological detection method is a radio-immunoassay (RIA), an enzyme linked immunosorbent assay (ELISA), a western blot, an immunohistochemical analysis, or fluorescence activated cell sorting (FACS).

9. The method of claim 1, whereby the vascular complication is cardiovascular complication that is hypercholesterolemia, angina, myocardial infarct, peripheral vascular disease, cerebrovascular disease or a combination thereof.

10. The method of claim 1, whereby the prognosis comprises determining the importance of reducing oxidative stress.

11. The method of claim 1, whereby the subject is diabetic.

12. The method of claim 1, wherein reverse cholesterol transport therapy is treatment with a cholesteryl ester transfer protein inhibitor.

13. The method of claim 12 wherein the cholesteryl ester transfer protein inhibitor is torcerapib.

14. The method of claim 1 wherein reverse cholesterol transport therapy is treatment with an antioxidant.

15. The method of claim 15 wherein the antioxidant is vitamin E.

16. The method of claim 14 wherein the antioxidant is glutathione peroxidase or its mimetic, isomer, metabolite, and/or salt thereof.

17. A method of treating, inhibiting or suppressing a vascular complication in a subject or reducing symptoms thereof, the method comprising the step of contacting the subject with an effective amount of a composition comprising glutathione peroxidase or its mimetic, isomer, metabolite, and/or salt therefore, and cholesteryl ester transfer protein inhibitor thereby treating, inhibiting or suppressing the vascular complication or symptoms thereof.

18. The method of claim 17, whereby said subject is diabetic.

19. The method of claim 16 or 17, whereby said glutathione peroxidase, a mimetic, isomer, a functional derivative, a synthetic analog, is represented by the compound of formula I:

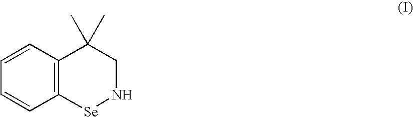

20. The method of claim 16 or 17, whereby said glutathione peroxidase or its mimetic, isomer, metabolite, and/or salt therefore, is benzisoselen-azoline or -azine derivatives represented by the following general formula II: 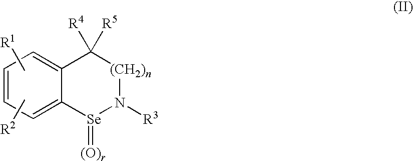
wherein R¹=R²=hydrogen; lower alkyl; OR⁶; —(CH₂)ₘNR⁶R⁷; —(CH₂)_{q}NH₂; —(CH₂)ₘ NHSO₂ (CH₂)₂ NH₂; —NO₂; —CN; —SO₃H; —N⁺(R⁵)₂O⁻; F; Cl; Br; I; —(CH₂)ₘR⁸; (CH₂)ₘ COR⁸; —S(O)NR⁶R⁷; —SO₂ NR⁶R⁷; —CO(CH₂)ₚCOR⁸; R⁹;
R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸;
R⁴=lower alkyl; aralkyl; substituted aralkyl; —(CH₂)ₚCOR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae: 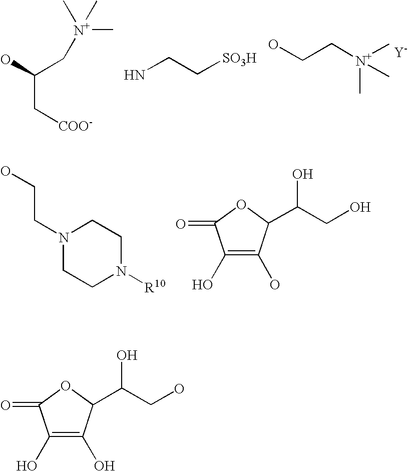
R¹⁰=hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=0, 1.

21. The method of claim 16 or 17, whereby the glutathione peroxidase or its mimetic, isomer, metabolite, and/or salt therefore is represented by the compound of formula III: 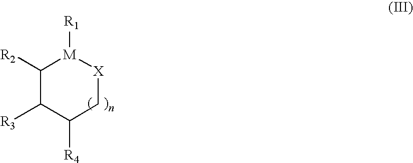
wherein,
the compound of formula 1 is a ring; and
X is O or NH
M is Se or Te
R₁ is oxygen; and forms an oxo complex with M; or
R₁ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, NR^{A}R^{B} or —SO₂R where R^{A} and R^{B} are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 member ring, substituted by an oxo group a to R₁, and said ring is fused to a second benzene ring, then M is Te.

22. The method of claim 21, whereby the compound of formula III is represented by the compound of formula IV-XIII or X: 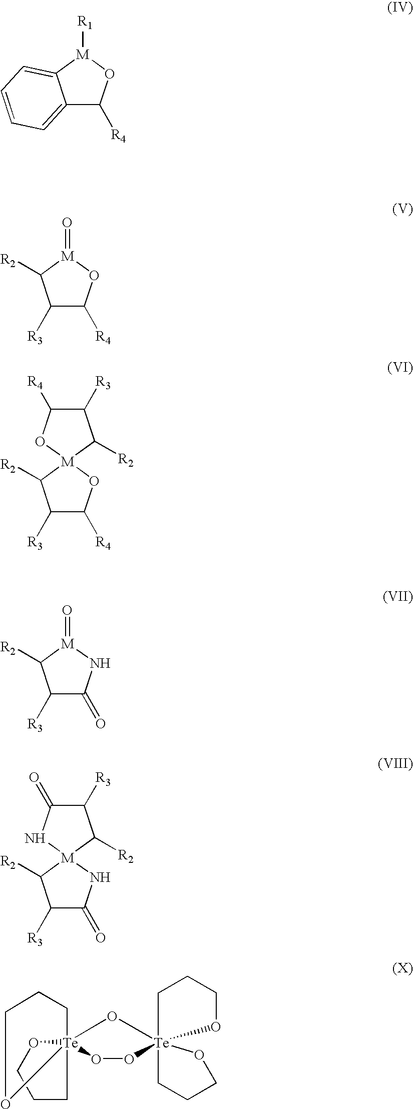
wherein, M, R₁, R₂, and R₃ are as described above; or

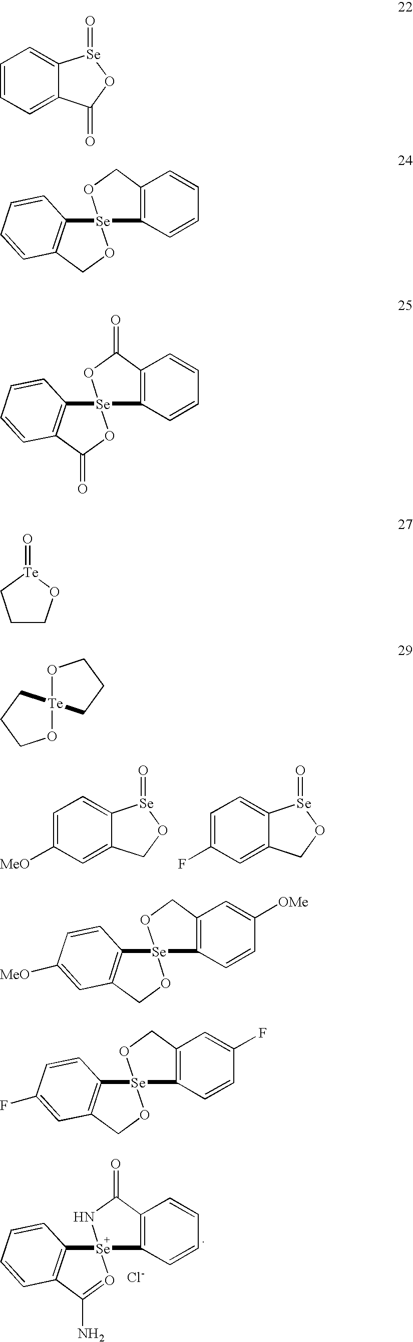

23. The method of claim 16 or 17, whereby the glutathione peroxidase or its mimetic, isomer, metabolite, and/or salt therefore is represented by the compound of formula IX: 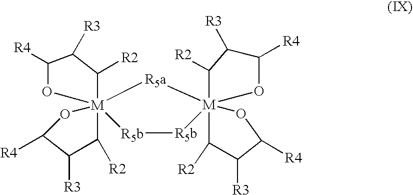
wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R_{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

24. The method of claim 17, whereby the vascular complication is cardiovascular complication that is hypercholesterolemia, angina, myocardial infarct, peripheral vascular disease, cerebrovascular disease or a combination thereof.

25. The method of claim 17, whereby the cholesteryl ester transfer protein inhibitor is torcetrapib.

26. The method of claim 17, preceded by the step of determining the Hp phenotype in said subject.

27. The method of claim 17, whereby the step of contacting is via oral, intravenous, intraarterial, intramuscular, subcutaneous, parenteral, transmucosal, transdermal, intracranial, or topical administration.

28. The method of claim 17, comprising contacting the subject with one or more additional agent, which is not glutathione peroxidase or its mimetic, isomer, metabolite, and/or salt therefore, nor a cholesteryl ester transfer protein inhibitor.

29. The method of claim 28, whereby the one or more additional agent not glutathione peroxidase or its mimetic, isomer, metabolite, and/or salt therefore, nor cholesteryl ester transfer protein inhibitor, is an aldosterone inhibitor, and angiotensin-converting enzyme, an antioxidant, an angiotensin receptor AT₁ blocker (ARB), an angiotensin II receptor antagonist, a calcium channel blocker, a diuretic, digitalis, a beta blocker, a statin, a cholestyramine, a NSAID, or a combination thereof.

30. The method of claim 17, further comprising removing haptoglobin.

31. A composition for treating a vascular complication in a subject comprising: a therapeutically effective amount of a composition comprising glutathione peroxidase or its mimetic, isomer, metabolite, and/or salt therefore and cholesteryl ester transfer protein inhibitor.

32. The composition of claim 31, wherein said glutathione peroxidase, its isomer, functional derivative, or synthetic analog and their combination is represented by the any one of the compounds of formula I-X, or their combination.

33. The composition of claim 31, wherein said composition is in a form suitable for oral, intravenous, intraarterial, intramuscular, subcutaneous, parenteral, transmucosal, transdermal, intracranial, or topical administration.

34. The composition of claim 31, wherein the vascular complication is cardiovascular complication that is hypercholesterolemia, angina, myocardial infarct, peripheral vascular disease, cerebrovascular disease or a combination thereof.

35. A method for correcting an abnormal or impaired reverse cholesterol transport in a diabetic patient, the method comprising the step of determining a haptoglobin phenotype of the diabetic patient, wherein ability to provide the correcting is greater in a patient having a haptoglobin 2-2 phenotype compared to patients having haptoglobin 1-2 phenotype or haptoglobin 1-1 phenotypes, and correcting the abnormal or impaired reverse cholesterol transport is achieved by administering an antioxidant.

36. The method of claim 35 wherein the antioxidant is vitamin E.

37. The method of claim 34 wherein the antioxidant is glutathione peroxidase or its mimetic, isomer, metabolite, and/or salt thereof.

38. A composition or pharmaceutical composition comprising: vitamin E or its derivative metabolite, or analog and their combination, and a glutathione peroxidase (GPx); its mimetic, isomer, functional derivative, synthetic analog, pharmaceutically acceptable salt or combination thereof.

39. A composition or pharmaceutical composition comprising: a statin; and a vitamin-E or its derivative metabolite, or analog and their combination.

40. A composition or pharmaceutical composition comprising a statin; and a GPx or its isomer, functional derivative, synthetic analog, pharmaceutically acceptable salt or combination thereof.
